SEQUENCE LISTING (104 sequences)

SEQ ID NO: 1 - protein, 120 aa, synthetic construct
  REGION 1..120: note = Feline/ human chimera anti-TGFB1,2,3 VH amino acid sequence
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 2 - DNA, 360 bp, synthetic construct
  misc_feature 1..360: note = Feline/ human chimera anti-TGFB1,2,3 VH nucleic acid sequence
  source 1..360: mol_type = other DNA
caggtgcagctggtgcagtccggagctgaggtgaagaagccaggctccagcgtgaaggtgtcttgcaaggcttccggctacaccttctcttccaacgtgatcagctgggtgagacaggctccaggacagggactggagtggatgggaggcgtgatccctatcgtggacatcgccaattacgctcagaggtttaagggccgggtgaccatcacagctgatgagtccacaagcaccacatatatggagctgagctctctgcgcagcgaggacaccgccgtgtactattgtgcttctacactgggcctggtgctggacgctatggactattggggccagggcaccctggtgacagtctcgagc

SEQ ID NO: 3 - protein, 6 aa, synthetic construct
  REGION 1..6: note = light chain anti-TGFB1,2,3 VL CDR1 amino acid sequence (CDR-K1)
RASQSL

SEQ ID NO: 4 - protein, 6 aa, synthetic construct
  REGION 1..6: note = light chain anti-TGFB1,2,3 VL CDR2 amino acid sequence (CDR-K2)
GASSRA

SEQ ID NO: 5 - protein, 9 aa, synthetic construct
  REGION 1..9: note = light chain anti-TGFB1,2,3 VL CDR3 amino acid sequence (CDR-K3)
QQYADSPIT

SEQ ID NO: 6 - protein, 108 aa, synthetic construct
  REGION 1..108: note = Felinized anti-TGFB1,2,3 VL1 amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTKLEIK

SEQ ID NO: 7 - protein, 108 aa, synthetic construct
  REGION 1..108: note = Felinized anti-TGFB1,2,3 VL2 amino acid sequence
EVVLTQSSAFLSRTLKEKATITCRASQSLGSSYLAWYQQKPNQAPKLLVKGASSRAPGAPSRFSGSGSGTDFTLTISSPEPEDAATYYCQQYADSPITFGQGTKLEIK

SEQ ID NO: 8 - protein, 108 aa, synthetic construct
  REGION 1..108: note = Felinized anti-TGFB1,2,3 VL3 amino acid sequence
EIQMTQSPSSLSASPGDRVTITCRASQSLGSSYLAWYQQKPGKVPKLLIYGASSRAPGVPSRFSGSGSGTDFTLTISSLEPEDAATYYCQQYADSPITFGQGTKLEIK

SEQ ID NO: 9 - protein, 108 aa, synthetic construct
  REGION 1..108: note = Felinized anti-TGFB1,2,3 VL4 amino acid sequence
DIVMTQTPSSVSGALGQRVTISCRASQSLGSSYLAWYQQVPGMAPKTIIYGASSRAPGVPDRFSGSKSGSTGTLTISGLQAEDEADYYCQQYADSPITFGQGTKLEIK

SEQ ID NO: 10 - protein, 114 aa, synthetic construct
  REGION 1..114: note = Felinized anti-TGFB1,2,3 VL5 amino acid sequence
QSRLTQPPSVSGSLGQRATISCRASQSLGSSYLAWHQQFPGMAPKTIIYGASSRAPGVPDRFSGSKSGNTGTLTITGLQAEDEADYYCQQYADSPITFGGGTHLTVLGLPKSAP

SEQ ID NO: 11 - protein, 114 aa, synthetic construct
  REGION 1..114: note = Felinized anti-TGFB1,2,3 VL6 amino acid sequence
QSVLTQPPSVSGSLGQRVTISCRASQSLGSSYLAWYQQVPGRAPRLLIYGASSRAPGIPDRFSGSKSGTSGSLTITGLEADDEADYYCQQYADSPITFGGGTHLTVLGLPKSAP

SEQ ID NO: 12 - protein, 107 aa, synthetic construct
  REGION 1..107: note = Felinized anti-TGFB1,2,3 VL7 amino acid sequence
DIVLTQPPSLSVNLGQTARITCRASQSLGSSYLAWYQQKPGQAPKLVTYGASSRAPGIPDRFSGTNSGITATLTISGARAEDEADYYCQQYADSPITFGQGTKLEIK

SEQ ID NO: 13 - protein, 108 aa, synthetic construct
  REGION 1..108: note = Felinized anti-TGFB1,2,3 VL8 amino acid sequence
DIVMTQSPGSLAGSPGQQVTMNCRASQSLGSSYLAWYQQKPGQHPELLIYGASSRAPGVPDRFSGSGSGTDFTLTISNLQAEDVANYYCQQYADSPITFGQGTKLEIK

SEQ ID NO: 14 - DNA, 324 bp, synthetic construct
  misc_feature 1..324: note = Felinized anti-TGFB1,2,3 VL1 nucleic acid sequence
  source 1..324: mol_type = other DNA
gagaccgtgctgacacagtctcctggcaccctgagcctgtctccaggagagagggccacactgtcctgcagggcttcccagagcctgggctccagctacctggcctggtatcagcagaagccaggccaggctcccaggctgctgatctacggagcctcttccagagctccaggcatccctgaccgcttctctggatccggaagcggcaccgacttcaccctgacaatcagcagactggagcccgaggacttcgccgtgtactattgtcagcagtatgctgattctcctatcacatttggccagggtaccaagctggagatcaaa

SEQ ID NO: 15 - DNA, 324 bp, synthetic construct
  misc_feature 1..324: note = Felinized anti-TGFB1,2,3 VL2 nucleic acid sequence
  source 1..324: mol_type = other DNA
gaggtggtgctgacccagtccagcgccttcctgtccagaacactgaaggagaaggccaccatcacatgccgcgcttctcagtccctgggctcttcctacctggcttggtatcagcagaagcctaaccaggccccaaagctgctggtgaagggagctagctctagggccccaggagctcccagccggttcagcggctctggctccggcaccgactttaccctgacaatctccagcccagagcctgaggacgctgctacctactattgtcagcagtacgccgattctcccatcacatttggccagggtaccaagctggagatcaaa

SEQ ID NO: 16 - DNA, 324 bp, synthetic construct
  misc_feature 1..324: note = Felinized anti-TGFB1,2,3 VL3 nucleic acid sequence
  source 1..324: mol_type = other DNA
gagatccagatgacccagtctccttccagcctgtctgcctccccaggcgacagggtgaccatcacatgccgggccagccagtctctgggctcttcctacctggcttggtatcagcagaagccaggcaaggtgcccaagctgctgatctacggagccagctctagagctccaggcgtgccttcccgcttctccggaagcggatctggcacagacttcaccctgacaatctccagcctggagccagaggacgctgctacctactattgtcagcagtatgctgatagccctatcacattcggccagggtaccaagctggagatcaaa

SEQ ID NO: 17 - DNA, 324 bp, synthetic construct
  misc_feature 1..324: note = Felinized anti-TGFB1,2,3 VL4 nucleic acid sequence
  source 1..324: mol_type = other DNA
gacatcgtgatgacccagacaccttccagcgtgtccggcgccctgggacagagggtgacaatcagctgcagggcttcccagagcctgggctcttcctacctggcctggtatcagcaggtgccaggcatggctcccaagaccatcatctacggagccagctctagagctccaggcgtgcctgaccgcttctctggatccaagagcggctctaccggcacactgaccatctctggcctgcaggccgaggacgaggctgattactattgtcagcagtatgccgattccccaatcacatttggccagggtaccaagctggagatcaaa

SEQ ID NO: 18 - DNA, 342 bp, synthetic construct
  misc_feature 1..342: note = Felinized anti-TGFB1,2,3 VL5 nucleic acid sequence
  source 1..342: mol_type = other DNA
cagtccaggctgacccagccacctagcgtgtctggctccctgggacagagggctacaatcagctgcagggctagccagtctctgggctccagctacctggcctggcaccagcagttccctggcatggctccaaagaccatcatctatggagcctcttccagagctccaggcgtgcccgaccgcttttccggaagcaagtctggcaacaccggcacactgaccatcacaggcctgcaggccgaggacgaggctgattactattgtcagcagtacgccgattctcccatcacattcggcggaggcacccatctgacagtgctgggcctgcctaagagcgctccc

SEQ ID NO: 19 - DNA, 342 bp, synthetic construct
  misc_feature 1..342: note = Felinized anti-TGFB1,2,3 VL6 nucleic acid sequence
  source 1..342: mol_type = other DNA
cagtctgtgctgacccagccacctagcgtgtctggctccctgggccagagagtgacaatctcctgcagggctagccagtctctgggctccagctacctggcctggtatcagcaggtgcctggaagggctccaaggctgctgatctacggagcctcttccagggctccaggcatccccgacagattctccggaagcaagtctggcacctccggcagcctgaccatcacaggcctggaggccgacgatgaggctgactactattgtcagcagtatgctgattcccccatcacatttggcggaggcacccacctgacagtgctgggactgcctaagagcgctccc

SEQ ID NO: 20 - DNA, 321 bp, synthetic construct
  misc_feature 1..321: note = Felinized anti-TGFB1,2,3 VL7 nucleic acid sequence
  source 1..321: mol_type = other DNA
gacatcgtgctgacacagccccctagcctgtctgtgaacctgggacagacagctaggatcacctgcagggcttcccagagcctgggctccagctacctggcctggtatcagcagaagcctggccaggctccaaagctggtgacatacggcgcctcttccagagctccaggcatccccgaccgcttctctggcaccaattccggcatcaccgccacactgaccatcagcggagccagggctgaggacgaggctgattactattgtcagcagtatgctgattctcccatcacctttggccagggtaccaagctggagatcaaa

SEQ ID NO: 21 - DNA, 324 bp, synthetic construct
  misc_feature 1..324: note = Felinized anti-TGFB1,2,3 VL8 nucleic acid sequence
  source 1..324: mol_type = other DNA
gacatcgtgatgacccagtcccctggcagcctggctggaagcccaggacagcaggtgacaatgaactgcagggccagccagtctctgggctccagctacctggcttggtatcagcagaagccaggccagcaccccgagctgctgatctacggagcctcttccagggctccaggcgtgcctgaccggttctccggaagcggatctggcaccgacttcaccctgacaatctctaacctgcaggccgaggacgtggctaattactattgtcagcagtatgctgattcccccatcacattcggccagggtaccaagctggagatcaaa

SEQ ID NO: 22 - protein, 10 aa, synthetic construct
  REGION 1..10: note = Felinized anti-TGFB1,2,3 VH CDR1 amino acid formula
  REGION 3..5: note = misc_feature - Xaa can be any naturally occurring amino acid
  REGION 9..10: note = misc_feature - Xaa can be any naturally occurring amino acid
GYXXXSNVXX

SEQ ID NO: 23 - protein, 10 aa, synthetic construct
  REGION 1..10: note = heavy chain anti-TGFB1,2,3 VH CDR1 amino acid sequence CDR #1 (CDR-H1)
GYTPSSNVIS

SEQ ID NO: 24 - protein, 10 aa, synthetic construct
  REGION 1..10: note = Felinized anti-TGFB1,2,3 VH CDR1 amino acid: VH8, VH2.8, VH3.8
GYTFSSNVIS

SEQ ID NO: 25 - protein, 10 aa, synthetic construct
  REGION 1..10: note = Felinized anti-TGFB1,2,3 VH CDR1 amino acid:, VH9
GYTFSSNVIH

SEQ ID NO: 26 - protein, 10 aa, synthetic construct
  REGION 1..10: note = Felinized anti-TGFB1,2,3 VH CDR1 amino acid: VH4
GYSFTSNVIS

SEQ ID NO: 27 - protein, 10 aa, synthetic construct
  REGION 1..10: note = Felinized anti-TGFB1,2,3 VH CDR1 amino acid: VH3
GFTYSSNVIS

SEQ ID NO: 28 - protein, 10 aa, synthetic construct
  REGION 1..10: note = Felinized anti-TGFB1,2,3 VH CDR1 amino acid: VH3.2
GYTFSSNVMH

SEQ ID NO: 29 - protein, 10 aa, synthetic construct
  REGION 1..10: note = Felinized anti-TGFB1,2,3 VH CDR1 amino acid: VH2.1
GYTFTSNVIS

SEQ ID NO: 30 - protein, 10 aa, synthetic construct
  REGION 1..10: note = Felinized anti-TGFB1,2,3 VH CDR1 amino acid: VH6.1
GYGFTSNVIS

SEQ ID NO: 31 - protein, 10 aa, synthetic construct
  REGION 1..10: note = Felinized anti-TGFB1,2,3 VH CDR1 amino acid: VH2.2
GYTFSSNVMH

SEQ ID NO: 32 - protein, 18 aa, synthetic construct
  REGION 1..18: note = Felinized anti-TGFB1,2,3 VH CDR2 amino acid: formula
  SITE 1: note = misc_feature - Xaa can be any naturally occurring amino acid
  SITE 10: note = misc_feature - Xaa can be any naturally occurring amino acid
  REGION 13..16: note = misc_feature - Xaa can be any naturally occurring amino acid
XVIPIVDIAXYAXXXXGR

SEQ ID NO: 33 - protein, 18 aa, synthetic construct
  REGION 1..18: note = heavy chain anti-TGFB1,2,3 VH CDR2 amino acid sequence CDR #2 (CDR-H2)
GVIPIVDIANYAQRPKGR

SEQ ID NO: 34 - protein, 18 aa, synthetic construct
  REGION 1..18: note = Felinized anti-TGFB1,2,3 VH CDR2 amino acid: VH8, VH4,3, VH2.1,VH6.1, VH2.8, VH3.8
GVIPIVDIANYAQRFKGR

SEQ ID NO: 35 - protein, 18 aa, synthetic construct
  REGION 1..18: note = Felinized anti-TGFB1,2,3 VH CDR2 amino acid: VH9
RVIPIVDIASYAQKFQGR

SEQ ID NO: 36 - protein, 18 aa, synthetic construct
  REGION 1..18: note = Felinized anti-TGFB1,2,3 VH CDR2 amino acid: VH3.2
YVIPIVDIAYYADSVKGR

SEQ ID NO: 37 - protein, 18 aa, synthetic construct
  REGION 1..18: note = Felinized anti-TGFB1,2,3VH CDR2 amino acid: VH2.2
SVIPIVDIATYARRFQGR

SEQ ID NO: 38 - protein, 13 aa, synthetic construct
  REGION 1..13: note = Felinized anti-TGFB1,2,3 VH CDR3 amino acid: formula
  SITE 2: note = misc_feature - Xaa can be any naturally occurring amino acid
AXTLGLVLDAMDY

SEQ ID NO: 39 - protein, 13 aa, synthetic construct
  REGION 1..13: note = heavy chain anti-TGFB1,2,3 VH CDR3 amino acid sequence CDR #3 (CDR-H3)
ASTLGLVLDAMDY

SEQ ID NO: 40 - protein, 13 aa, synthetic construct
  REGION 1..13: note = Felinized anti-TGFB1,2,3 VH CDR3 for VH8, VH9 amino acidsequence, ARRLGLVLDAMDY
ARRLGLVLDAMDY

SEQ ID NO: 41 - protein, 13 aa, synthetic construct
  REGION 1..13: note = Felinized anti-TGFB1,2,3 VH CDR3 forVH3, VH 3.2, VH 2.2 aminoacid sequence
ARTLGLVLDAMDY

SEQ ID NO: 42 - protein, 120 aa, synthetic construct
  REGION 1..120: note = Caninized anti-TGFB1,2,3 VH2 amino acid sequence
EVQLVQSAAEVKKPGASVKVSCKTSGYTFTSNVISWIQRAPGAGLDWMGGVIPIVDIANYAQRFKGRVTLTADTSTGTTYTELSSLRAEDTAVYYCARTLGLVLDAMDYWGQGTLVTVSS

SEQ ID NO: 43 - protein, 119 aa, synthetic construct
  REGION 1..119: note = Caninized anti-TGFB1,2,3 VH6 amino acid sequence
EVQLVQSGAEVKKPGASVKVSCKTSGYTFISNVISWVRQAPGAGLDWMGGVIPIVDIANYAQRFKGRVTLTADTSTSTAYMELSSLRAGDIAVYYCATLGLVLDAMDYWGQGTLVTVSS

SEQ ID NO: 44 - protein, 120 aa, synthetic construct
  REGION 1..120: note = Caninized anti-TGFB1,2,3 VH3 amino acid sequence
EVQLVQSAAEVKKPGASVKVSCKTSGYIFISNVISWVQQAPGAGLEWMGGVIPIVDIANYAQRFKGRVTLTADTSTNTVYMELSNLRTEDTAVYYCARTLGLVLDAMDYWGQGTLVTVSS

SEQ ID NO: 45 - protein, 120 aa, synthetic construct
  REGION 1..120: note = Caninized anti-TGFB1,2,3 VH4 amino acid sequence
EVQLVESGGDLVKPAGSLRLSCVASGFTFSSNVISWVRQAPGKGLQWVGGVIPIVDIANYAQRFKGRFTISRDNAKNTLYLQMNSLRVEDTAVYYCTRTLGLVLDAMDYWGQGTLVTVSS

SEQ ID NO: 46 - protein, 120 aa, synthetic construct
  REGION 1..120: note = Caninized anti-TGFB1,2,3 VH4.11 amino acid sequence
QVQLVQSGAEVKKPGSSVKVSCKASGYTFSSNVISWVRQAPGKGLQWVGGVIPIVDIANYAQRFKGRVTLTADTSTNTVYMELSNLRTEDTAVYYCARTLGLVLDAMDYWGQGTLVTVSS

SEQ ID NO: 47 - protein, 120 aa, synthetic construct
  REGION 1..120: note = Caninized anti-TGFB1,2,3 VH5.10 amino acid sequence
EVQLVESGGDLVKPGGSLRLSCVASGFTFSSNVISWVRQAPGQGLEWMGGVIPIVDIANYAQRFKGRVTLTADTSTNTVYMELSNLRTEDTAVYYCARTLGLVLDAMDYWGQGTLVTVSS

SEQ ID NO: 48 - protein, 120 aa, synthetic construct
  REGION 1..120: note = Caninized anti-TGFB1,2,3 VH4.5 amino acid sequence
EVQLVESGGDLVKPAGSLRLSCVASGYTFSSNVMSWVRQAPGKGLQWVGYVIPIVDIANYADAVKGRFTISRDNAKNTLYLQMNSLRVEDTAVYYCTRTLGLVLDAMDYWGQGTLVTVSS

SEQ ID NO: 49 - protein, 120 aa, synthetic construct
  REGION 1..120: note = Caninized anti-TGFB1,2,3 VH3.1 amino acid sequence
DVQLVESGGDLVKPGGSLRLTCVASGYTFSSNVMHWVRQAPGKGLQWVAYVIPIVDIAYYADSVKGRFTISIDNSKNTLYLQMNSLKTEDTATYYCARTLGLVLDAMDYWGQGTLVTVSS

SEQ ID NO: 50 - protein, 120 aa, synthetic construct
  REGION 1..120: note = Caninized anti-TGFB1,2,3 VH3.5 amino acid sequence
EVQLVQSAAEVKKPGASVKVSCKTSGYIFISNVISWVQQAPGAGLEWMGGVIPIVDIANYAQRFKGRVTLTADTSTNTVYMELSNLRTEDTAVYYCASTLGLVLDAMDYWGQGTLVTVSS

SEQ ID NO: 51 - protein, 120 aa, synthetic construct
  REGION 1..120: note = Caninized anti-TGFB1,2,3 VH3.6 amino acid sequence
EVQLVQSAAEVKKPGASVKVSCKTSGYIFISNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 52 - protein, 120 aa, synthetic construct
  REGION 1..120: note = Caninized anti-TGFB1,2,3 VH4.7 amino acid sequence
QVQLVQSGAEVKKPGSSVKVSCKASGYTFSSNVISWVRQAPGKGLQWVGGVIPIVDIANYAQRFKGRVTITADESTSTTYMELSSLRSEDTAVYYCASTLGLVLDAMDYWGQGTLVTVSS

SEQ ID NO: 53 - protein, 120 aa, synthetic construct; identical to SEQ ID NO: 46
  REGION 1..120: note = Caninized anti-TGFB1,2,3 VH4.11 amino acid sequence

SEQ ID NO: 54 - protein, 120 aa, synthetic construct
  REGION 1..120: note = Caninized anti-TGFB1,2,3 VH5.6 amino acid sequence
EVQLVESGGDLVKPGGSLRLSCVASGFTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 55 - DNA, 359 bp, synthetic construct
  misc_feature 1..359: note = Caninized anti-TGFB1,2,3 VH2 nucleic acid sequence
  source 1..359: mol_type = other DNA
gaggtgcagctggtgcagagcgccgctgaggtgaagaagccaggcgcctctgtgaaggtgtcctgcaagaccagcggctacaccttcacatctaacgtgatctcctggatccagagggctccaggagctggactggactggatgggcggcgtgatccctatcgtggatatcgccaattacgctcagaggtttaagggccgggtgaccctgacagctgacacctctacaggcaccacatatacagagctgtccagcctgagagccgaggataccgccgtgtactattgtgctcgcacactgggactggtgctggacgctatggattattggggccagggcaccctggtgacagtctcgag

SEQ ID NO: 56 - DNA, 356 bp, synthetic construct
  misc_feature 1..356: note = Caninized anti-TGFB1,2,3 VH6 nucleic acid sequence
  source 1..356: mol_type = other DNA
gaggtgcagctggtgcagtccggagctgaggtgaagaagccaggagctagcgtgaaggtgtcttgcaagacatccggctacaccttcatcagcaacgtgatctcttgggtgagacaggctccaggagctggactggactggatgggcggcgtgatccctatcgtggatatcgccaattacgctcagaggtttaagggaagggtgaccctgacagctgacacctctacatccaccgcttatatggagctgtccagcctgagggctggcgatatcgccgtgtactattgtgccacactgggcctggtgctggacgctatggattattggggccagggcacactggtgaccgtctcgag

SEQ ID NO: 57 - DNA, 359 bp, synthetic construct
  misc_feature 1..359: note = Caninized anti-TGFB1,2,3 VH3 nucleic acid sequence
  source 1..359: mol_type = other DNA
gaggtgcagctggtgcagagcgccgctgaggtgaagaagccaggcgcctccgtgaaggtgagctgcaagacatctggctacatcttcatctccaacgtgatcagctgggtgcagcaggctccaggagctggactggagtggatgggcggcgtgatccctatcgtggacatcgccaattacgctcagaggtttaagggccgggtgaccctgacagccgatacctctacaaacaccgtgtatatggagctgtccaatctgagaacagaggacaccgccgtgtactattgtgctcgcaccctgggactggtgctggacgctatggattattggggccagggcacactggtgaccgtctcgag

SEQ ID NO: 58 - DNA, 359 bp, synthetic construct
  misc_feature 1..359: note = Caninized anti-TGFB1,2,3 VH4 nucleic acid sequence
  source 1..359: mol_type = other DNA
gaggtgcagctggtggagagcggaggcgatctggtgaagccagctggctctctgaggctgtcctgcgtggctagcggcttcaccttttccagcaacgtgatctcttgggtgagacaggctccaggcaagggactgcagtgggtgggaggcgtgatccctatcgtggacatcgccaactacgctcagaggttcaagggccggtttaccatctctagggataacgctaagaatacactgtatctgcagatgaactccctgagagtggaggacacagccgtgtactattgtacccgcacactgggcctggtgctggacgctatggattactggggccagggcaccctggtgacagtctcgag

SEQ ID NO: 59 - DNA, 359 bp, synthetic construct
  misc_feature 1..359: note = Caninized anti-TGFB1,2,3 VH4.11 nucleic acid sequence
  source 1..359: mol_type = other DNA
caggtgcagctggtgcagtctggcgccgaggtgaagaagccaggctccagcgtgaaggtgagctgcaaggcttctggctacacattctcttccaacgtgatctcctgggtgaggcaggctccaggcaagggactgcagtgggtgggaggcgtgatccctatcgtggacatcgccaattacgctcagaggtttaagggccgggtgaccctgacagctgatacctccacaaacaccgtgtatatggagctgagcaatctgagaacagaggacaccgccgtgtactattgtgctcgcaccctgggactggtgctggacgctatggactattggggccagggcacactggtgaccgtctcgag

SEQ ID NO: 60 - DNA, 359 bp, synthetic construct
  misc_feature 1..359: note = Caninized anti-TGFB1,2,3 VH5.10 nucleic acid sequence
  source 1..359: mol_type = other DNA
gaggtgcagctggtggagagcggaggcgatctggtgaagccaggaggctctctgaggctgtcctgcgtggctagcggcttcacattttccagcaacgtgatctcttgggtgagacaggctccaggacagggactggagtggatgggaggcgtgatccctatcgtggacatcgccaattacgctcagaggttcaagggccgggtgaccctgacagccgatacctctacaaacaccgtgtatatggagctgtccaatctgagaacagaggacaccgccgtgtactattgtgctcgcaccctgggactggtgctggacgctatggattactggggccagggcacactggtgaccgtctcgag

SEQ ID NO: 61 - DNA, 359 bp, synthetic construct
  misc_feature 1..359: note = Caninized anti-TGFB1,2,3 VH4.5 nucleic acid sequence
  source 1..359: mol_type = other DNA
gaggtgcagctggtggagagcggaggcgacctggtgaagccagctggctctctgaggctgtcctgcgtggctagcggctacaccttctccagcaacgtgatgtcttgggtgagacaggctccaggcaagggactgcagtgggtgggctacgtgatccctatcgtggacatcgccaactatgccgatgctgtgaagggcaggtttaccatctctcgggacaacgctaagaatacactgtacctgcagatgaactccctgagagtggaggatacagccgtgtactattgtacccgcacactgggcctggtgctggacgctatggattattggggccagggcaccctggtgacagtctcgag

SEQ ID NO: 62 - DNA, 359 bp, synthetic construct
  misc_feature 1..359: note = Caninized anti-TGFB1,2,3 VH3.1 nucleic acid sequence
  source 1..359: mol_type = other DNA
gaggtgcagctggtgcagagcgccgctgaggtgaagaagccaggcgcctccgtgaaggtgagctgcaagacatctggctacatcttcatctccaacgtgatcagctgggtgcagcaggctccaggagctggactggagtggatgggcggcgtgatccctatcgtggacatcgccaattacgctcagaggtttaagggccgggtgaccctgacagccgatacctctacaaacaccgtgtatatggagctgtccaatctgaggacagaggacaccgccgtgtactattgtgcttctaccctgggcctggtgctggacgctatggattattggggccagggcacactggtgaccgtctcgag

SEQ ID NO: 63 - DNA, 359 bp, synthetic construct
  misc_feature 1..359: note = Caninized anti-TGFB1,2,3 VH3.5 nucleic acid sequence
  source 1..359: mol_type = other DNA
gaggtgcagctggtgcagagcgccgctgaggtgaagaagccaggcgcctctgtgaaggtgtcctgcaagacaagcggctacaccttctccagcaacgtgatgcactgggtgcagcaggctccaggagctggactggagtggatgggctgggtcatccctatcgtggacatcgcctcttacgctcagaagtttcagggcagggtgaccctgacagccgatacctctacaaacaccgtgtatatggagctgtccaatctgaggacagaggacaccgccgtgtactattgtgctcgggatctgggcctggtgctggacgctatggattattggggccagggcacactggtgaccgtctcgag

SEQ ID NO: 64 - DNA, 359 bp, synthetic construct
  misc_feature 1..359: note = Caninized anti-TGFB1,2,3 VH3.6 nucleic acid sequence
  source 1..359: mol_type = other DNA
gaggtgcagctggtgcagtctgccgctgaggtgaagaagccaggcgccagcgtgaaggtgtcttgcaagacctccggctacatcttcatcagcaacgtgatctcttgggtgagacaggctccaggacagggactggagtggatgggaggcgtgatccctatcgtggacatcgccaattacgctcagaggtttaagggccgggtgaccatcacagccgatgagtctacatccaccacatatatggagctgtccagcctgcgctccgaggacaccgccgtgtactattgtgctagcacactgggcctggtgctggacgctatggattattggggccagggcaccctggtgacagtctcgag

SEQ ID NO: 65 - DNA, 359 bp, synthetic construct
  misc_feature 1..359: note = Caninized anti-TGFB1,2,3 VH4.7 nucleic acid sequence
  source 1..359: mol_type = other DNA
caggtgcagctggtgcagtccggagctgaggtgaagaagccaggctccagcgtgaaggtgtcttgcaaggcttccggctacaccttctcttccaacgtgatcagctgggtgagacaggctccaggcaagggactgcagtgggtgggaggcgtgatccctatcgtggacatcgccaattacgctcagaggtttaagggccgggtgaccatcacagctgatgagtccacaagcaccacatatatggagctgagctctctgcgcagcgaggacaccgccgtgtactattgtgcttctacactgggcctggtgctggacgctatggactattggggccagggcaccctggtgacagtctcgag

SEQ ID NO: 66 - DNA, 359 bp, synthetic construct
  misc_feature 1..359: note = Caninized anti-TGFB1,2,3 VH5.6 nucleic acid sequence
  source 1..359: mol_type = other DNA
gaggtgcagctggtggagtctggaggcgatctggtgaagccaggaggctccctgagactgagctgcgtggcttctggcttcaccttttccagcaacgtgatctcctgggtgcgccaggctccaggacagggactggagtggatgggaggcgtgatccctatcgtggacatcgccaattacgctcagaggttcaagggccgggtgaccatcacagccgatgagagcacatctaccacatatatggagctgtcttccctgaggtccgaggacaccgccgtgtactattgtgctagcacactgggcctggtgctggacgctatggattactggggccagggcaccctggtgacagtctcgag

SEQ ID NO: 67 - protein, 108 aa, synthetic construct
  REGION 1..108: note = Caninized anti-TGFB1,2,3 VL1 amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 68 - protein, 108 aa, synthetic construct
  REGION 1..108: note = Caninized anti-TGFB1,2,3 VL2 amino acid sequence
ETVLTQSPGTLSLSPGERATLSCRASQSLGSSYLAWYQQKPGQAPRLLIYGASSRAPGVPARFSGSGSGTDFTLTISSLEPEDFAVYYCQQYADSPITFGQGTKLEIK

SEQ ID NO: 69 - protein, 108 aa, synthetic construct
  REGION 1..108: note = Caninized anti-TGFB1,2,3 VL3 amino acid sequence
EIVMTQSPASLSLSQEEKVTITCRASQSLGSSYLAWYQQKPGQAPKLLIYGASSRAPGVPSRFSGSGSGTDFSFTISSLEPEDVAVYYCQQYADSPITFGQGTKLEIK

SEQ ID NO: 70 - protein, 108 aa, synthetic construct
  REGION 1..108: note = Caninized anti-TGFB1,2,3 VL4 amino acid sequence
EAVMTQTPLSLAVTPGELATISCRASQSLGSSYLAWYLQKPGQTPRPLIYGASSRAPGVSDRFSGSGSGTDFTLKISRVEAEDVGVYYCQQYADSPITFGQGTRLEIK

SEQ ID NO: 71 - DNA, 309 bp, synthetic construct
  misc_feature 1..309: note = Caninized anti-TGFB1,2,3 VL1 nucleic acid sequence
  source 1..309: mol_type = other DNA
gagaccgtgctgacacagtctcctggcaccctgagcctgtctccaggagagagggccacactgtcctgcagggcttcccagagcctgggctccagctacctggcctggtatcagcagaagccaggccaggctcccaggctgctgatctacggagcctcttccagagctccaggcatccctgaccgcttctctggatccggaagcggcaccgacttcaccctgacaatcagcagactggagcccgaggacttcgccgtgtactattgtcagcagtatgctgattctcctatcacatttggccagggtacc

SEQ ID NO: 72 - DNA, 309 bp, synthetic construct
  misc_feature 1..309: note = Caninized anti-TGFB1,2,3 VL 2 nucleic acid sequence
  source 1..309: mol_type = other DNA
gagaccgtgctgacacagagccctggcaccctgtccctgagcccaggagagagggccacactgtcttgccgggcttctcagtccctgggctccagctacctggcctggtatcagcagaagccaggccaggctcccaggctgctgatctacggagcctcttccagagctccaggcgtgcctgctcgcttcagcggatctggctccggcaccgactttaccctgacaatcagctctctggagcccgaggacttcgccgtgtactattgtcagcagtatgctgattcccctatcacatttggccagggtacc

SEQ ID NO: 73 - DNA, 309 bp, synthetic construct
  misc_feature 1..309: note = Caninized anti-TGFB1,2,3 VL3 nucleic acid sequence
  source 1..309: mol_type = other DNA
gagatcgtgatgacccagagcccagcctccctgagcctgtctcaggaggagaaggtgaccatcacatgcagggcttcccagagcctgggctccagctacctggcctggtatcagcagaagccaggccaggctcccaagctgctgatctacggagcctcttccagggctccaggcgtgccttctcggttctctggatccggaagcggcaccgacttctcctttacaatcagctctctggagcctgaggacgtggccgtgtactattgtcagcagtatgctgattctccaatcacatttggccagggtacc

SEQ ID NO: 74 - DNA, 309 bp, synthetic construct
  misc_feature 1..309: note = Caninized anti-TGFB1,2,3 VL 4 nucleic acid sequence
  source 1..309: mol_type = other DNA
gaggccgtgatgacccagacaccactgtccctggctgtgaccccaggagagctggccacaatcagctgcagagctagccagtctctgggctccagctacctggcctggtatctgcagaagcctggacagaccccccgccctctgatctacggagcctcttccagggctccaggcgtgtctgaccggttctccggaagcggatctggcaccgacttcaccctgaagatctccagggtggaggctgaggacgtgggcgtgtactattgtcagcagtatgctgatagccccatcacattcggccagggtacc

SEQ ID NO: 75 - protein, 335 aa, Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 76 - DNA, 1005 bp, Felis catus
  source 1..1005: mol_type = other DNA
gcctccaccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccggccgtcctgcaggcctcggggctgtactctctcagcagcatggtgacagtgccctccagcaggtggctcagtgacaccttcacctgcaacgtggcccacccgcccagcaacaccaaggtggacaagaccgtgcgcaaaacagaccacccaccgggacccaaaccctgcgactgtcccaaatgcccaccccctgagatgcttggaggaccgtccatcttcatcttccccccaaaacccaaggacaccctctcgatttcccggacgcccgaggtcacatgcttggtggtggacttgggcccagatgactccgatgtccagatcacatggtttgtggataacacccaggtgtacacagccaagacgagtccgcgtgaggagcagttcaacagcacctaccgtgtggtcagtgtcctccccatcctacaccaggactggctcaaggggaaggagttcaagtgcaaggtcaacagcaaatccctcccctcccccatcgagaggaccatctccaaggccaaaggacagccccacgagccccaggtgtacgtcctgcctccagcccaggaggagctcagcaggaacaaagtcagtgtgacctgcctgatcaaatccttccacccgcctgacattgccgtcgagtgggagatcaccggacagccggagccagagaacaactaccggacgaccccgccccagctggacagcgacgggacctacttcgtgtacagcaagctctcggtggacaggtcccactggcagaggggaaacacctacacctgctcggtgtcacacgaagctctgcacagccaccacacacagaaatccctcacccagtctccgggtaaa

SEQ ID NO: 77 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGAPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 78 - DNA, 1005 bp, Canis familiaris
  source 1..1005: mol_type = other DNA
gcctcaacaactgctcctagcgtgtttcccctggcccctagctgcggaagtacctcaggcagcacagtggccctggcttgtctggtgtctggatatttccctgagccagtgaccgtgagttggaacagcggctctctgacctccggggtgcacacatttccatctgtgctgcagtctagtggcctgtactccctgtcaagcatggtgactgtgccttcctctaggtggccatcagaaactttcacctgcaacgtggcccatcccgccagcaagaccaaagtggacaagcccgtgcctaaaagggagaatggaagggtgccaagaccacctgattgccctaagtgtccagctccagaagcggcgggagcaccaagcgtgttcatctttccacccaagcccaaagacacactgctgattgctagaactcccgaggtgacctgcgtggtggtggacctggatccagaggaccccgaagtgcagatctcctggttcgtggatgggaagcagatgcagacagccaaaactcagcctcgggaggaacagtttaacggaacctatagagtggtgtctgtgctgccaattggacaccaggactggctgaagggcaaacagtttacatgcaaggtgaacaacaaggccctgcctagtccaatcgagaggactatttcaaaagctaggggacaggctcatcagccttccgtgtatgtgctgcctccatcccgggaggaactgtctaagaacacagtgagtctgacttgtctgatcaaagatttctttccccctgacattgatgtggagtggcagagcaatgggcagcaggagccagaatccaagtacagaaccacaccaccccagctggacgaagatggctcctatttcctgtacagtaagctgtcagtggacaaatctaggtggcagcgcggggatacctttatctgcgccgtgatgcacgaggctctgcacaatcattacacacaagaaagtctgtcacatagccccggcaag

SEQ ID NO: 79 - protein, 120 aa, synthetic construct
  REGION 1..120: note = GC1008 VH amino acid sequence
QVQLVQSGAEVKKPGSSVKVSCTASGYTPSSNVISNVRQAPGQGLENNGGVIPIVDIANYAQRPKGRVTITADESTSTTYHELSSLRSEDTAVYYCASTLGLVLDAMDYHGQGTLVTVSS

SEQ ID NO: 80 - protein, 113 aa, synthetic construct
  REGION 1..113: note = GC1008 VL amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAA

SEQ ID NO: 81 - protein, 106 aa, Canis familiaris
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 82 - DNA, 324 bp, Canis familiaris
  source 1..324: mol_type = other DNA
cggaacgacgcccagcccgccgtgtacctgttccagcccagccccgatcagctgcacaccggcagcgcttcagtcgtctgcctgctgaacagcttctaccccaaggacatcaacgtgaagtggaaggtggacggcgtgatccaggacaccggcatccaggaaagcgtcaccgagcaggacaaggacagcacctacagcctgagcagcaccctgaccatgtccagcaccgagtacctgagccacgagctgtatagctgcgagatcacccacaagagcctgcctagcaccctgatcaagagcttccagcggagcgagtgctagtag

SEQ ID NO: 83 - protein, 110 aa, Felis catus
RSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFQRSECQRE

SEQ ID NO: 84 - DNA, 336 bp, Felis catus
  source 1..336: mol_type = other DNA
cggagtgatgctcagccatctgtctttctcttccaaccatctctggacgagttacatacaggaagtgcctctatcgtgtgcatattgaatgacttctaccccaaagaggtcaatgtcaagtggaaagtggatggcgtagtccaaaacaaaggcatccaggagagcaccacagagcagaacagcaaggacagcacctacagcctcagcagcaccctgacgatgtccagtacggagtaccaaagtcatgaaaagttctcctgcgaggtcactcacaagagcctggcctccaccctcgtcaagagcttccagaggagcgagtgtcagagagagtgatga

SEQ ID NO: 85 - protein, 120 aa, synthetic construct
  REGION 1..120: note = Felinized anti-TGFB1,2,3 VH3 amino acid sequence
DVQLVESGGDLVKPGGSLRLTCVASGFTYSSNVISWVRQAPGKGLQWVAGVIPIVDIANYAQRFKGRFTISIDNSKNTLYLQMNSLKTEDTATYYCARTLGLVLDAMDYWGQGTLVTVSS

SEQ ID NO: 86 - protein, 120 aa, synthetic construct
  REGION 1..120: note = Felinized anti-TGFB1,2,3 VH4 amino acid sequence
QVLLVQSGAEVRKPEASVEIFCKASGYSFTSNVISWVRQAPAQGFEWMGGVIPIVDIANYAQRFKGRLTLTADTSTNTAYMELSSLRSTDTAVYYCASTLGLVLDAMDYWGQGTLVTVSS

SEQ ID NO: 87 - protein, 120 aa, synthetic construct
  REGION 1..120: note = Felinized anti-TGFB1,2,3 VH8 amino acid sequence
QVLLVQSGAEVKKPGASVKIFCKASGYTFSSNVISWLRQAPEQGLEWMGGVIPIVDIANYAQRFKGRLTLTADTSTNTVYMELSSLRSADTAMYYCARRLGLVLDAMDYWGQGTLVTVSS

SEQ ID NO: 88 - protein, 120 aa, synthetic construct
  REGION 1..120: note = Felinized anti-TGFB1,2,3 VH9 amino acid sequence
QVLLVQSGAEVKKPGASVKIFCKASGYTFSSNVIHWLRQAPEQGLEWMGRVIPIVDIASYAQKFQGRLTLTADTSTNTVYMELSSLRSADTAMYYCARRLGLVLDAMDYWGQGTLVTVSS

SEQ ID NO: 89 - protein, 120 aa, synthetic construct; identical to SEQ ID NO: 49
  REGION 1..120: note = Felinized anti-TGFB1,2,3 VH3.2 amino acid sequence

SEQ ID NO: 90 - protein, 120 aa, synthetic construct
  REGION 1..120: note = Felinized anti-TGFB1,2,3 VH2.1 amino acid sequence
QVLLVQSGADVRKPGASVKIFCKASGYTFTSNVISWVRQTPAQGFEWMGGVIPIVDIANYAQRFKGRLVLTADTSTNTAYMELRSLKSADTAVYYCASTLGLVLDAMDYWGQGSLVTVSS

SEQ ID NO: 91 - protein, 120 aa, synthetic construct
  REGION 1..120: note = Felinized anti-TGFB1,2,3 VH6.1 amino acid sequence
QVLLVQSGAEVRTPGASVKIFCKASGYGFTSNVISWVRQSPAQGLEWMGGVIPIVDIANYAQRFKGRLTLTADTSTDTAYMELSSLRSADTAMYYCASTLGLVLDAMDYWGQGTLVTVSS

SEQ ID NO: 92 - protein, 120 aa, synthetic construct
  REGION 1..120: note = Felinized anti-TGFB1,2,3 VH2.2 amino acid sequence
QVLLVQSGADVRKPGASVKIFCKASGYTFSSNVMHWVRQTPAQGFEWMGSVIPIVDIATYARRFQGRLVLTADTSTNTAYMELRSLKSADTAVYYCARTLGLVLDAMDYWGQGSLVTVSS

SEQ ID NO: 93 - protein, 120 aa, synthetic construct
  REGION 1..120: note = Felinized anti-TGFB1,2,3 VH2.8 amino acid sequence
QVQLVQSGAEVKKPGSSVKVSCKASGYTFSSNVISWVRQTPAQGFEWMGGVIPIVDIANYAQRFKGRLVLTADTSTNTAYMELRSLKSADTAVYYCASTLGLVLDAMDYWGQGTLVTVSS

SEQ ID NO: 94 - protein, 120 aa, synthetic construct
  REGION 1..120: note = Felinized anti-TGFB1,2,3 VH3.8 amino acid sequence
QVQLVQSGAEVKKPGSSVKVSCKASGYTFSSNVISWVRQAPGKGLQWVAGVIPIVDIANYAQRFKGRFTISIDNSKNTLYLQMNSLKTEDTATYYCASTLGLVLDAMDYWGQGTLVTVSS

SEQ ID NO: 95 - DNA, 360 bp, synthetic construct
  misc_feature 1..360: note = Felinized anti-TGFB1,2,3 VH3 nucleic acid sequence
  source 1..360: mol_type = other DNA
gacgtgcagctggtggagtccggaggcgatctggtgaagccaggaggcagcctgagactgacctgcgtggcttctggcttcacatactccagcaacgtgatcagctgggtgcgccaggctccaggcaagggactgcagtgggtggctggcgtgatccctatcgtggacatcgccaactacgctcagaggttcaagggccggtttaccatctctatcgataactccaagaatacactgtatctgcagatgaactccctgaagaccgaggacaccgccacatactattgtgctaggacactgggcctggtgctggacgctatggattattggggccagggcaccctggtgacagtctcgagc

SEQ ID NO: 96 - DNA, 360 bp, synthetic construct
  misc_feature 1..360: note = Felinized anti-TGFB1,2,3 VH4 nucleic acid sequence
  source 1..360: mol_type = other DNA
caggtgctgctggtgcagtctggcgccgaggtgagaaagccagaggcttccgtggagatcttctgcaaggcctctggctactcctttacaagcaacgtgatctcctgggtgcgccaggctccagctcagggattcgagtggatgggcggcgtgatccctatcgtggacatcgccaactacgctcagaggtttaagggccggctgaccctgacagccgatacctccacaaataccgcttatatggagctgtccagcctgaggagcacagacaccgccgtgtactattgtgcttctaccctgggcctggtgctggacgctatggattattggggccagggcacactggtgaccgtctcgagc

SEQ ID NO: 97 - DNA, 360 bp, synthetic construct
  misc_feature 1..360: note = Felinized anti-TGFB1,2,3 VH8 nucleic acid sequence
  source 1..360: mol_type = other DNA
caggtgctgctggtgcagtctggcgccgaggtgaagaagccaggcgctagcgtgaagatcttctgcaaggcctctggctacacattttccagcaacgtgatctcctggctgaggcaggctccagagcagggactggagtggatgggaggcgtgatccctatcgtggacatcgccaactacgctcagagattcaagggccgcctgaccctgacagccgatacctccacaaataccgtgtatatggagctgtcttccctgagaagcgccgacaccgctatgtactattgtgctaggcggctgggactggtgctggacgctatggattattggggccagggcacactggtgaccgtctcgagc

SEQ ID NO: 98 - DNA, 360 bp, synthetic construct
  misc_feature 1..360: note = Felinized anti-TGFB1,2,3 VH9 nucleic acid sequence
  source 1..360: mol_type = other DNA
caggtgctgctggtgcagtctggcgccgaggtgaagaagccaggcgctagcgtgaagatcttctgcaaggcctctggctacacattttccagcaacgtgatccactggctgagacaggctccagagcagggactggagtggatgggacgcgtgatccctatcgtggacatcgcctcctacgctcagaagttccagggcaggctgaccctgacagccgatacctccacaaataccgtgtatatggagctgtcttccctgagaagcgccgacaccgctatgtactattgtgctaggcggctgggactggtgctggacgctatggattattggggccagggcacactggtgaccgtctcgagc

SEQ ID NO: 99 - DNA, 360 bp, synthetic construct
  misc_feature 1..360: note = Felinized anti-TGFB1,2,3 VH3.2 nucleic acid sequence
  source 1..360: mol_type = other DNA
gacgtgcagctggtggagtctggaggcgatctggtgaagccaggaggcagcctgaggctgacctgcgtggcttctggctacacattctccagcaacgtgatgcactgggtgcggcaggctccaggcaagggactgcagtgggtggcttatgtgatccctatcgtggacatcgcctactatgctgattccgtgaagggcaggttcaccatctctatcgacaactccaagaatacactgtacctgcagatgaatagcctgaagaccgaggataccgccacatactattgtgctcgcacactgggcctggtgctggacgctatggattattggggccagggcaccctggtgacagtctcgagc

SEQ ID NO: 100 - DNA, 360 bp, synthetic construct
  misc_feature 1..360: note = Felinized anti-TGFB1,2,3 VH2.1 nucleic acid sequence
  source 1..360: mol_type = other DNA
caggtgctgctggtgcagtctggcgccgatgtgagaaagccaggcgctagcgtgaagatcttctgcaaggcctctggctacacctttacatctaacgtgatctcctgggtgcgccagacaccagctcagggattcgagtggatgggaggcgtgatccctatcgtggacatcgccaactacgctcagaggtttaagggccggctggtgctgaccgctgatacctccacaaataccgcttatatggagctgaggtccctgaagagcgccgacacagccgtgtactattgtgcctccaccctgggactggtgctggacgctatggattattggggccagggcagcctggtgacagtctcgagc

SEQ ID NO: 101 - DNA, 363 bp, synthetic construct
  misc_feature 1..363: note = Felinized anti-TGFB1,2,3 VH6.1 nucleic acid sequence
  source 1..363: mol_type = other DNA
caggtgctgctggtgcagtctggcgccgaggtgagaacaccaggcgctagcgtgaagatcttctgcaaggcctctggctacggctttacctctaacgtgatctcctgggtgcgccagtccccagctcagggactggagtggatgggaggcgtgatccctatcgtggacatcgccaattacgctcagaggttcaagggccggctgaccctgacagctgacacctccacagataccgcttatatggagctgtccagcctgaggtccgccgatacagctatgtactattgtgccagcaccctgggactggtgctggacgctatggattattggggccagggcacactggtgaccgtggtctcgagc

SEQ ID NO: 102 - DNA, 360 bp, synthetic construct
  misc_feature 1..360: note = Felinized anti-TGFB1,2,3 VH2.2 nucleic acid sequence
  source 1..360: mol_type = other DNA
caggtgctgctggtgcagtctggcgccgatgtgaggaagccaggcgctagcgtgaagatcttctgcaaggcctctggctacacattttccagcaacgtgatgcactgggtgagacagacccccgctcagggcttcgagtggatgggctccgtgatccctatcgtggacatcgccacatacgctaggcggtttcagggcaggctggtgctgaccgccgataccagcacaaataccgcttatatggagctgagatctctgaagtccgccgacacagccgtgtactattgtgctcgcaccctgggactggtgctggacgctatggattattggggccagggctccctggtgacagtctcgagc

SEQ ID NO: 103 - DNA, 360 bp, synthetic construct
  misc_feature 1..360: note = Felinized anti-TGFB1,2,3 VH2.8 nucleic acid sequence
  source 1..360: mol_type = other DNA
caggtgcagctggtgcagagcggagctgaggtgaagaagccaggctccagcgtgaaggtgtcctgcaaggctagcggctacacattctcttccaacgtgatctcttgggtgagacagaccccagctcagggatttgagtggatgggaggcgtgatccctatcgtggacatcgccaactacgctcagaggttcaagggccggctggtgctgacagccgatacctctacaaataccgcttatatggagctgcgcagcctgaagtctgccgacacagccgtgtactattgtgcctccaccctgggactggtgctggacgctatggattattggggccagggcacactggtgaccgtctcgagc

SEQ ID NO: 104 - DNA, 360 bp, synthetic construct
  misc_feature 1..360: note = Felinized anti-TGFB1,2,3 VH3.8 nucleic acid sequence
  source 1..360: mol_type = other DNA
caggtgcagctggtgcagagcggagctgaggtgaagaagccaggctccagcgtgaaggtgagctgcaaggcttctggctacaccttctcttccaacgtgatctcttgggtgaggcaggctccaggcaagggactgcagtgggtggctggcgtgatccctatcgtggacatcgccaactacgctcagaggttcaagggccggtttaccatctccatcgataacagcaagaatacactgtatctgcagatgaactccctgaagaccgaggacaccgccacatactattgtgcttccacactgggcctggtgctggacgctatggactattggggccagggcaccctggtgacagtctcgagc

(19) United States
(12) Patent Application Publication
(10) Pub. No.: US 2023/0192834 A1
(43) Pub. Date: Jun. 22, 2023

(54) ANTI- TGFB1,2,3 ANTIBODIES AND THERAPEUTIC USES THEREOF

(71) Applicant: Zoetis Services LLC, Parsippany, NJ (US)

(72) Inventors: Lisa Marie BERGERON, Boulder, CO (US); Henry Luis CAMPOS, Kalamazoo, MI (US)

(21) Appl. No.: 17/935,590

(22) Filed: Sep. 27, 2022

Related U.S. Application Data
(60) Provisional application No. 63/248,679, filed on Sep. 27, 2021.

(51) Int. Cl. C07K 16/22; C12N 15/63

(52) U.S. Cl. CPC C07K 16/22; C12N 15/63; C07K 2317/565; C07K 2317/20

(57) ABSTRACT

The present disclosure encompasses novel anti-TGFβ1,2,3 antibodies and polynucleotides encoding the same. The disclosure further provides use of the novel antibodies and/or nucleotide of the invention for the treatment and/or prevention of TGFβ-related disorders, particularly in for the management of fibrosis related disorders in canines and felines.

### FIELD OF THE DISCLOSURE

The present application relates to monoclonal antibodies, methods of their production, and therapeutic uses of those antibodies. In certain embodiments, the monoclonal antibodies are directed toward Transforming Growth Factor-Beta (TGFβ, TGFB, TGFb or TGFbeta). In other embodiments, the antibodies are chimeric or speciated antibodies. In other embodiments method of treatments comprising the antibodies of the invention are disclosed.

### REFERENCE TO AN ELECTRONIC SEQUENCE LISTING

The contents of the electronic sequence listing (ZP000385A.xml; size: 150 kilobytes: and Date of Creation: Sep. 22, 2022) is herein incorporated by reference in its entirety.

### BACKGROUND OF THE INVENTION

Transforming Growth Factor-Beta (TGFB, TGFβ or TGF beta, as used interchangeably herein) is a cytokine that controls many key cellular functions including proliferation, differentiation, survival, migration and epithelial mesenchymal transition. It is a member of a superfamily of 38 cytokines that include TGFβ, bone morphogenetic proteins (BMP), growth differentiation factors, inhibins, and activins. TGFβ proteins regulate diverse biologic processes such as extracellular matrix formation, wound healing, embryonic development, bone development, hematopoiesis, immune and inflammatory responses and malignant transformation. Deregulation of TGFβ leads to pathological conditions that include birth defects, cancer, chronic inflammation, autoimmune and fibrotic diseases.

TGFβ has three known isoforms, TGFβ1,2, and 3. All three isoforms are initially translated as a pro-peptide. The isoforms are synthesized as large precursor proteins (pro-TGFβ) forming dimeric complexes in the endoplasmic reticulum and are subsequently cleaved near the carboxy-terminus to yield mature 112-amino acid polypeptides which share 60-80% conservation across the three TGFβ isoforms. The mature TGFβ dimer remains associated with the cleaved latency peptide portion of the precursor as an inactive latent complex. Newly synthesized TGFβ bound to the latency-associated peptide (LAP) forming a small latent complex (SLC) is biologically inactive and cannot bind to its receptor, TGFβRII. Through the formation of disulfide bonds this complex loosely binds to a latent TGFβ binding protein (LTBP) to form a large latent complex (LLC). TGFβ is then secreted in a latent state and is stored in the extracellular matrix (ECM). Activation of TGFβ involves release from the latent complex following exposure to a number of different factors, including integrins, proteases, metalloproteinases, reactive oxygen species (ROS), plasmin, and acid, that allow binding to its cell surface receptors for initiation of TGFβ signaling.

TGFβ1,2 and 3 are pleiotropic in their function and are expressed in different patterns across cell and tissue types. They have similar in vitro activities, but individual knockouts in specific cell types suggest non-identical roles in vivo despite their shared ability to bind to the same receptor (Akhurst et al., Nat Rev Drug Discov (2012) 11 (10): 790-811). Upon TGFβ binding to TGFβRII, the constituitive kinase activity of the receptor phosphorylates and activates TGFβRI which in turn phosphorylates SMAD2/3 allowing for association with SMAD4. This complex localizes to the nucleus and serve as a transcription factor for TGFβ responsive genes. In addition to this canonical signaling cascade, a non-canonical pathway transmits signals through other factors including p38, MAPK, PI3K, AKT, JUN, JNK and NK-KB. The end result is a crosstalk of all of these signalling pathways that integrate the state and environment of the cell.

Many severe diseases are linked to malfunctions of the TGFβ induced signaling pathway. The present invention is directed towards the potential treatment of both canine and feline chronic kidney disease (CKD). CKD involves a loss of functional kidney tissue due to a prolonged, progressive process. Dramatic changes in kidney structure may be seen, although structural and functional changes in the kidney are only loosely correlated. Disease is usually present for many months or years before it becomes clinically apparent, and it is invariably irreversible. Although congenital disease results in a transient increase in prevalence in animals >3 years old, the prevalence increases with advancing age from 5-6 years onward. In geriatric populations CKD affects as many as 10% of dogs and 40-80% of cats. In the field of veterinary medicine there is a distinct and unmet need to treat CKD in both dogs and cats which is suggested to be a condition influenced by an overproduction of TGFβ proteins.

### SUMMARY OF THE INVENTION

The present invention provides novel anti-Transforming Growth Factor Beta (TGFB, TGFbeta, TGFb or TGFβ as defined and used interchangeably herein) antigen binding proteins (antibody, antibody fragment, antagonist antibody, as defined and used interchangeably herein) that binds to TGFβ1, TGFβ2 and TGFβ3, particularly canine TGFβ1, TGFβ2 and TGFβ3 and feline TGFβ1, TGFβ2 and TGFβ3, sequences well known to one of skill in the art. The antigen binding protein of the invention blocks the biological activity of TGFβ1, TGFβ2 and TGFβ3 from preventing the binding of TGFβ1, TGFβ2 and TGFβ3 to its receptor and prevents activation of the pathways associated with binding. Additionally, the present invention provides that the antagonist action of the antibody of the invention prevents and/or treats a TGFβ related disorder, as defined herein. The invention further provides nucleotides that encode the antigen binding protein of the invention as well as the production of vectors and host cells. The invention further provides methods of making and using said antibody/antigen binding protein as well as methods of treatment of treating TGFβ disorders in canines and in felines by administering the antibody of the invention.

In one aspect, the invention provides an antibody that specifically binds to canine TGFβ1, TGFβ2 and TGFβ3 and comprising heavy chain complimentarity determining regions (CDRs) comprising SEQ ID NO. 23; SEQ ID NO. 33 and SEQ ID NO. 39 and light chain complimentarity determining regions (CDRs) comprising SEQ ID NO. 3, SEQ ID NO. 4 and SEQ ID NO. 5 and variants thereof, wherein said antibody further comprises a canine IgGB constant region comprising an amino acid sequence having at least about 95% sequence identity to SEQ ID NO. 77.

In one aspect, the invention provides an antibody that specifically binds to canine TGFβ1, TGFβ2 and TGFβ3 and comprising a heavy chain variable region (VH) having at least about 95% sequence identity to the amino acid sequences selected from the group consisting of: SEQ ID NOs 42-54; and a light chain variable region (VL) having at least about 95% sequence identity to the amino acid sequences selected from the group consisting of: SEQ ID NOs 67-70.

In one embodiment the antibody comprises a caninized antibody.

In one aspect, the invention provides an antibody that specifically binds to feline TGFβ1, TGFβ2 and TGFβ3 comprising heavy chain complimentarity determining regions (CDRs) SEQ ID NO. 23, SEQ ID NO. 33 and SEQ ID NO. 39 and light chain complimentarity determining regions (CDRs) SEQ ID NO. 3, SEQ ID NO. 4 and SEQ ID NO. 5 and variants thereof, wherein said antibody comprises a feline IgG1a constant region comprising an amino acid sequence having at least about 95% sequence identity to SEQ ID NO. 75.

In one aspect, the invention provides an antibody that specifically binds to feline TGFβ1, TGFβ2 and TGFβ3 comprising

a. a variable heavy (VH) chain comprising:
i. a Complimentary Determining Region 1 (CDR1) comprising an amino acid sequence having at least about 95% sequence identity to the amino acid sequence comprising SEQ ID NO. 22 (G-Y-X1-X2-X3-S—N-V-X4-X5), wherein:
   X1 comprises T or G;
   X2 comprises F or P;
   X3 comprises S or T;
   X4 comprises M or I;
   X5 comprises H or S; and
ii. a Complimentary Determining Region 2 (CDR2) comprising an amino acid sequence having at least about 95% sequence identity to the amino acid sequence comprising SEQ ID NO. 32 (X6-V-I-P-I-V-D-I-A-X7-Y-A-X8-X9-X10-X11-G-R), wherein:
   X6 comprises G, Y or S;
   X7 comprises N, Y or T;
   X8 comprises Q or R;
   X9 comprises R, or S;
   X10 comprises F or V;
   X11 comprises K or Q; and
iii. a Complimentary Determining Region 3 (CDR3) comprising an amino acid sequence having at least about 95% sequence identity to the amino acid sequence comprising: SEQ ID NO: 38 (A-X12-T-L-G-L-V-L-D-A-M-D-Y) wherein:
   X12 comprises R or S; and

b. a light chain variable region (VL) comprising:
i. a Complimentary Determining Region 1 (CDR1) comprising an amino acid sequence having at least about 95% sequence identity to the amino acid sequence comprising SEQ ID NO. 3;
ii. a Complimentary Determining Region 2 (CDR2) comprising an amino acid sequence having at least about 95% sequence identity to the amino acid sequence comprising SEQ ID NO. 4;
iii. a Complimentary Determining Region 3 (CDR3) comprising an amino acid sequence having at least about 95% sequence identity to the amino acid sequence comprising SEQ ID NO: 5; and
   any variant thereof having one or more conservative amino acid substitutions; wherein said antibody comprises a feline IgG1a constant region comprising an amino acid sequence having at least about 95% sequence identity to SEQ ID NO. 75.

In one embodiment, the invention provides an antibody that comprises a heavy chain variable region (VH) having at least about 95% sequence identity to the amino acid sequences selected from the group consisting of: SEQ ID NO:87-94; and a light chain variable region (VL) having at least about 95% sequence identity to the amino acid sequences selected from the group consisting of: SEQ ID NO: 6-13 and any variant thereof having one or more conservative amino acid substitutions.

In one embodiment, the invention provides an antibody that comprises a felinized antibody.

In one embodiment, the invention provides an antibody that is selected from the group consisting of: a monoclonal antibody; a single chain antibody, a tetrameric antibody, a tetravalent antibody, a multispecific antibody, a domain-specific antibody, a domain-deleted antibody, a fusion protein, an ScFc fusion protein, an Fab fragment, an Fab′ fragment, an F(ab′)₂ fragment, an Fv fragment, an ScFv fragment, an Fd fragment, a single domain antibody, a dAb fragment, a small modular immunopharmaceutical (SMIP) a nanobody, and IgNAR molecule. In one embodiment the antibody comprises a monoclonal antibody.

In one or more embodiments the invention provides and antibody for use in treating a TGFβ-related disorder. In one embodiment the TGFβ-related disorder is selected from the group consisting of fibrosis disorder, connective tissue disorder, bone disorders and cell proliferation disorders. In one embodiment the TGFβ-related disorder comprises a fibrosis disorder. In one embodiment the fibrosis disorder is selected from the group consisting of kidney fibrosis/chronic kidney disease; pulmonary fibrosis; cirrhosis of the liver; glial scarring; and systemic sclerosis/scleroderma. In one embodiment the TGFβ related disorder is kidney fibrosis/chronic kidney disease.

In one aspect, the invention provides a pharmaceutical composition comprising therapeutically effective amount of the antibody of any one of claims 1-14 and a pharmaceutically acceptable carrier.

In one aspect, the invention provides a method of treating a subject for a TGFβ related disorder by administering to said subject a therapeutic amount of the pharmaceutical composition of the invention. In one embodiment the subject comprises a canine. In one embodiment the subject comprises a feline. In one or more embodiments the TGFβ related disorder is selected from the group consisting of: fibrosis disorder, connective tissue disorder, bone disorders and cell proliferation disorders. In one embodiment the TGFβ related disorder comprises a fibrosis disorder. In one embodiment the fibrosis disorder is selected from the group consisting of kidney fibrosis/chronic kidney disease; pulmonary fibrosis; cirrhosis of the liver; glial scarring; and systemic sclerosis/scleroderma. In one embodiment the TGFβ disorder is kidney fibrosis/chronic kidney disease.

In one aspect, the invention provides a method of inhibiting TGFβ1, 2 and 3 activity in a subject by administering the pharmaceutical composition of the invention. In one embodiment the subject comprises a canine. In one embodiment the subject comprises a feline.

In one aspect, the invention provides an isolated nucleic acid sequence having at least about 95% sequence identity to the nucleic acid sequence encoding one or more antibodies of the invention and any variants thereof having one or more nucleic acid substitutions resulting in conservative amino acid substitutions.

In one aspect, the invention provides an isolated nucleic acid sequence encoding one or more antibodies of the invention wherein said sequence comprises a nucleotide sequence encoding the VH having 95% sequence identity to SEQ ID NO. 55-66 and a nucleotide sequence encoding the VL having 95% sequence identity to SEQ ID NO. 71-74 and any variants thereof having one or more nucleic acid substitutions resulting in conservative amino acid substitutions.

In one aspect, the invention provides an isolated nucleic acid sequence encoding one or more antibodies of the invention wherein said sequence comprises a nucleotide sequence encoding the VH having 95% sequence identity to SEQ ID NO. 97-104 and a nucleotide sequence encoding the VL having 95% sequence identity to SEQ ID NO. 14-21 and any variants thereof having one or more nucleic acid substitutions resulting in conservative amino acid substitutions.

In one aspect, the invention provides a vector comprising one or more of the nucleic acid sequences of the invention.

In one aspect, the invention provides a host cell comprising one or more of the nucleic acid sequences of the invention.

In one aspect, the invention provides a host cell comprising the vector of the invention.

In one aspect, the invention provides a host cell that produces the antibody of the invention.

In one aspect, the invention provides a method of producing the antibody of the invention by culturing the host cell of the invention under conditions that result in production of the antibody and isolating the antibody from the host cell or culture medium of the host cell.

### BRIEF DESCRIPTION OF THE DRAWINGS

FIG. 1 represents the general structure of a native mouse immunoglobulin G (IgG) highlighting the antigen binding site.

FIG. 2 is a schematic representation of the general structure of one embodiment of a mouse: canine IgG.

FIG. 3 represents a heterochimeric molecule.

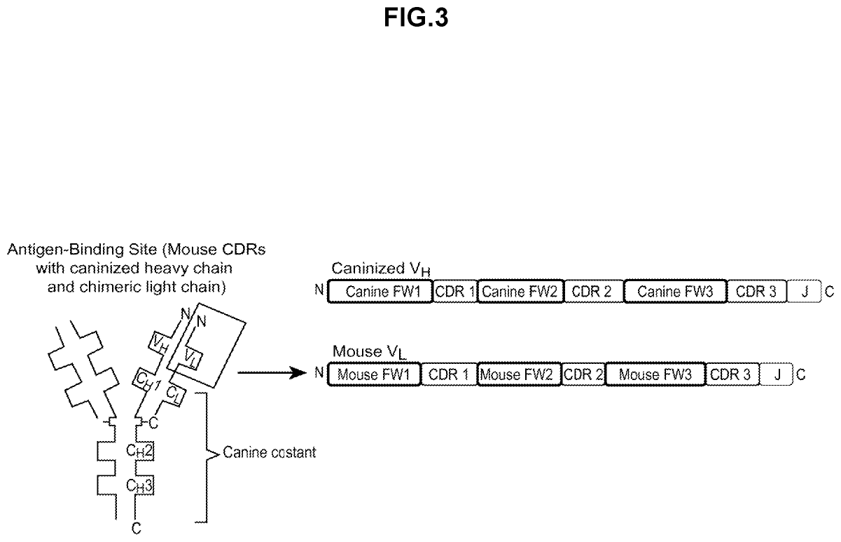

FIG. 4 represents speciation or caninization of a mouse IgG.

### BRIEF DESCRIPTION OF THE SEQUENCES

SEQ ID NO. 1 Feline/human chimera anti-TGFβ1,2,3 VH amino acid sequence.
SEQ ID NO. 2 Feline/human chimera anti-TGFβ1,2,3 VH nucleic acid sequence.
SEQ ID NO. 3 anti-TGFβ1,2,3 VL CDR1 amino acid sequence.
SEQ ID NO. 4 anti-TGFβ1,2,3 VL CDR1 amino acid sequence.
SEQ ID NO. 5 anti-TGFβ1,2,3 VL CDR1 amino acid sequence.
SEQ ID NO. 6 Felinized anti-TGFβ1,2,3 VL1 amino acid sequence.
SEQ ID NO. 7 Felinized anti-TGFβ1,2,3 VL2 amino acid sequence.
SEQ ID NO. 8 Felinized anti-TGFβ1,2,3 VL3 amino acid sequence.
SEQ ID NO. 9 Felinized anti-TGFβ1,2,3 VL4 amino acid sequence.
SEQ ID NO. 10 Felinized anti-TGFβ1,2,3 VL5 amino acid sequence.
SEQ ID NO. 11 Felinized anti-TGFβ1,2,3 VL6 amino acid sequence.
SEQ ID NO. 12 Felinized anti-TGFβ1,2,3 VL7 amino acid sequence.
SEQ ID NO. 13 Felinized anti-TGFβ1,2,3 VL8 amino acid sequence.
SEQ ID NO. 14 Felinized anti-TGFβ1,2,3 VL1 nucleic acid sequence.
SEQ ID NO. 15 Felinized anti-TGFβ1,2,3 VL2 nucleic acid sequence.
SEQ ID NO. 16 Felinized anti-TGFβ1,2,3 VL3 nucleic acid sequence.
SEQ ID NO. 17 Felinized anti-TGFβ1,2,3 VL4 nucleic acid sequence.
SEQ ID NO. 18 Felinized anti-TGFβ1,2,3 VL5 nucleic acid sequence.
SEQ ID NO. 19 Felinized anti-TGFβ1,2,3 VL6 nucleic acid sequence.
SEQ ID NO. 20 Felinized anti-TGFβ1,2,3 VL7 nucleic acid sequence.
SEQ ID NO. 21 Felinized anti-TGFβ1,2,3 VL8 nucleic acid sequence.
SEQ ID NO. 22 anti-TGFβ1,2,3 VH CDR1 amino acid formula.
SEQ ID NO. 23 anti-TGFβ1,2,3 VH CDR1 amino acid sequence.
SEQ ID NO. 24 Felinized anti-TGFβ1,2,3 VH CDR1 amino acid: VH8, VH2.8, VH3.8.
SEQ ID NO. 25 Felinized anti-TGFβ1,2,3 VH CDR1 amino acid: VH9.
SEQ ID NO. 26 Felinized anti-TGFβ1,2,3 VH CDR1 amino acid: VH4.
SEQ ID NO. 27 Felinized anti-TGFβ1,2,3 VH CDR1 amino acid: VH3.
SEQ ID NO. 28 Felinized anti-TGFβ1,2,3 VH CDR1 amino acid: VH3.2.
SEQ ID NO. 29 Felinized anti-TGFβ1,2,3 VH CDR1 amino acid: VH2.1.
SEQ ID NO. 30 Felinized anti-TGFβ1,2,3 VH CDR1 amino acid: VH6.1.
SEQ ID NO. 31 Felinized anti-TGFβ1,2,3 VH CDR1 amino acid: VH2.2.
SEQ ID NO. 32 Felinized anti-TGFβ1,2,3 VH CDR2 amino acid: formula.
SEQ ID NO. 33 anti-TGFβ1,2,3 VH CDR2 amino acid.
SEQ ID NO. 34 Felinized anti-TGFβ1,2,3 VH CDR2 amino acid: VH8, VH4,3, VH2.1, VH 6.1, VH2.8, VH3.8.
SEQ ID NO. 35 Felinized anti-TGFβ1,2,3 VH CDR2 amino acid: VH9.
SEQ ID NO. 36 Felinized anti-TGFβ1,2,3 VH CDR2 amino acid: VH3.2.
SEQ ID NO. 37 Felinized anti-TGFβ1,2,3VH CDR2 amino acid: VH2.2.
SEQ ID NO. 38 Felinized anti-TGFβ1,2,3 VH CDR3 amino acid: formula.
SEQ ID NO. 39 anti-TGFβ1,2,3 VH CDR3 amino acid.
SEQ ID NO. 40 Felinized anti-TGFβ1,2,3 VH CDR3 for VH8, VH9 amino acid sequence.
SEQ ID NO. 41 Felinized anti-TGFβ1,2,3 VH CDR3 for VH3, VH 3.2, VH 2.2 amino acid sequence.
SEQ ID NO. 42 Caninized anti-TGFβ1,2,3 VH2 amino acid sequence.
SEQ ID NO. 43 Caninized anti-TGFβ1,2,3 VH6 amino acid sequence.
SEQ ID NO. 44 Caninized anti-TGFβ1,2,3 VH3 amino acid sequence.
SEQ ID NO. 45 Caninized anti-TGFβ1,2,3 VH4 amino acid sequence.
SEQ ID NO. 46 Caninized anti-TGFβ1,2,3 VH4.11 amino acid sequence.
SEQ ID NO. 47 Caninized anti-TGFβ1,2,3 VH5.10 amino acid sequence.
SEQ ID NO. 48 Caninized anti-TGFβ1,2,3 VH4.5 amino acid sequence.
SEQ ID NO. 49 Caninized anti-TGFβ1,2,3 VH3.1 amino acid sequence.
SEQ ID NO. 50 Caninized anti-TGFβ1,2,3 VH3.5 amino acid sequence.
SEQ ID NO. 51 Caninized anti-TGFβ1,2,3 VH3.6 amino acid sequence.
SEQ ID NO. 52 Caninized anti-TGFβ1,2,3 VH4.7 amino acid sequence.
SEQ ID NO. 53 Caninized anti-TGFβ1,2,3 VH4.11 amino acid sequence.
SEQ ID NO. 54 Caninized anti-TGFβ1,2,3 VH5.6 amino acid sequence.
SEQ ID NO. 55 Caninized anti-TGFβ1,2,3 VH2 nucleic acid sequence.
SEQ ID NO. 56 Caninized anti-TGFβ1,2,3 VH6 nucleic acid sequence.
SEQ ID NO. 57 Caninized anti-TGFβ1,2,3 VH3 nucleic acid sequence.
SEQ ID NO. 58 Caninized anti-TGFβ1,2,3 VH4 nucleic acid sequence.
SEQ ID NO. 59 Caninized anti-TGFβ1,2,3 VH4.11 nucleic acid sequence.
SEQ ID NO. 60 Caninized anti-TGFβ1,2,3 VH5.10 nucleic acid sequence.
SEQ ID NO. 61 Caninized anti-TGFβ1,2,3 VH4.5 nucleic acid sequence.
SEQ ID NO. 62 Caninized anti-TGFβ1,2,3 VH3.1 nucleic acid sequence.
SEQ ID NO. 63 Caninized anti-TGFβ1,2,3 VH3.5 nucleic acid sequence.
SEQ ID NO. 64 Caninized anti-TGFβ1,2,3 VH3.6 nucleic acid sequence.
SEQ ID NO. 65 Caninized anti-TGFβ1,2,3 VH4.7 nucleic acid sequence.
SEQ ID NO. 66 Caninized anti-TGFβ1,2,3 VH5.6 nucleic acid sequence.
SEQ ID NO. 67 Caninized anti-TGFβ1,2,3 VL1 amino acid sequence.
SEQ ID NO. 68 Caninized anti-TGFβ1,2,3 VL2 amino acid sequence.
SEQ ID NO. 69 Caninized anti-TGFβ1,2,3 VL3 amino acid sequence.
SEQ ID NO. 70 Caninized anti-TGFβ1,2,3 VL4 amino acid sequence.
SEQ ID NO. 71 Caninized anti-TGFβ1,2,3 VL1 nucleic acid sequence.
SEQ ID NO. 72 Caninized anti-TGFβ1,2,3 VL 2 nucleic acid sequence.
SEQ ID NO. 73 Caninized anti-TGFβ1,2,3 VL3 nucleic acid sequence.
SEQ ID NO. 74 Caninized anti-TGFβ1,2,3 VL 4 nucleic acid sequence.
SEQ ID NO. 75: Feline Heavy Chain Constant Region IgG1a amino acid sequence.
SEQ ID NO. 76: Feline Heavy Chain Constant Region IgG1a nucleic acid sequence.
SEQ ID NO. 77: Canine Heavy Chain Constant Region IgGB amino acid sequence.
SEQ ID NO. 78 Canine Heavy Chain Constant Region IgGB nucleic acid sequence.
SEQ ID NO. 79 GC1008 VH amino acid sequence.
SEQ ID NO. 80 GC1008 VL amino acid sequence.
SEQ ID NO. 81 Canine light chain constant region amino acid sequence.
SEQ ID NO. 82 Canine light chain constant region nucleic acid sequence.
SEQ ID NO. 83 Feline light chain constant region amino acid sequence.
SEQ ID NO. 84 Feline light chain constant region nucleic acid sequence.
SEQ ID NO. 85 Felinized anti-TGFβ1,2,3 VH3 amino acid sequence.
SEQ ID NO. 86 Felinized anti-TGFβ1,2,3 VH4 amino acid sequence.
SEQ ID NO. 87 Felinized anti-TGFβ1,2,3 VH8 amino acid sequence.
SEQ ID NO. 88 Felinized anti-TGFβ1,2,3 VH9 amino acid sequence.
SEQ ID NO. 89 Felinized anti-TGFβ1,2,3 VH3.2 amino acid sequence.
SEQ ID NO. 90 Felinized anti-TGFβ1,2,3 VH2.1 amino acid sequence.
SEQ ID NO. 91 Felinized anti-TGFβ1,2,3 VH6.1 amino acid sequence.
SEQ ID NO. 92 Felinized anti-TGFβ1,2,3 VH2.2 amino acid sequence.
SEQ ID NO. 93 Felinized anti-TGFβ1,2,3 VH2.8 amino acid sequence.
SEQ ID NO. 94 Felinized anti-TGFβ1,2,3 VH3.8 amino acid sequence.
SEQ ID NO. 95 Felinized anti-TGFβ1,2,3 VH3 nucleic acid sequence.
SEQ ID NO. 96 Felinized anti-TGFβ1,2,3 VH4 nucleic acid sequence.
SEQ ID NO. 97 Felinized anti-TGFβ1,2,3 VH8 nucleic acid sequence.
SEQ ID NO. 98 Felinized anti-TGFβ1,2,3 VH9 nucleic acid sequence.
SEQ ID NO. 99 Felinized anti-TGFβ1,2,3 VH3.2 nucleic acid sequence.
SEQ ID NO. 100 Felinized anti-TGFβ1,2,3 VH2.1 nucleic acid sequence.
SEQ ID NO. 101 Felinized anti-TGFβ1,2,3 VH6.1 nucleic acid sequence.
SEQ ID NO. 102 Felinized anti-TGFβ1,2,3 VH2.2 nucleic acid sequence.
SEQ ID NO. 103 Felinized anti-TGFβ1,2,3 VH2.8 nucleic acid sequence.
SEQ ID NO. 104 Felinized anti-TGFβ1,2,3 VH3.8 nucleic acid sequence.

### DETAILED DESCRIPTION OF THE INVENTION

The invention disclosed herein provides anti-TGFβ antibody/antibody fragments (terms used interchangeably) that bind TGFβ1 and TGFβ2 and TGFβ proteins with high affinity and specificity. The invention further provides antibodies and polypeptides that also bind to the TGFβ1, 2 and 3 proteins or polypeptides described herein that are variants of said antibodies as well as methods of making and using said antibodies. In some embodiments, the invention also provides polynucleotides encoding said antibodies and/or polypeptides. The invention disclosed herein also provides methods for preventing and/or treating a TGFβ related disorder selected from the group of fibrosis disorder, connective tissue disorder, bone disorders and cell proliferation disorders by administration of a therapeutically effective amount of the anti-TGFβ1,2, and 3 antibodies and the respective variants of the invention described herein.

### General Techniques and Definitions

It should be understood that this invention is not limited to the particular methodology, protocols, and reagents etc., described herein and as such may vary. The terminology used herein is only for the purpose of describing particular embodiments and is not intended to limit the scope of the present invention which is defined solely by the claims. Unless otherwise defined, scientific and technical terms used in connection with the invention as described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art and are not limited to a single description. It is well known in the art that different techniques may be substituted for what is described.

All patents and other publications identified are expressly incorporated herein by reference for the purpose of describing and disclosing, for example, the methodologies described in such publications that might be used in connection with the present invention. These publications are provided solely for their disclosure prior to the filing date of the present application

Standard techniques are used for recombinant DNA, oligonucleotide and polynucleotide synthesis, tissue culture, transfection and transformation of cells, among many other commonly used techniques well known to one of skill in the art. General techniques well known to those of skill in the art are performed per manufacturer's specifications or as commonly accomplished in the art or as described herein.

Before describing the present invention in detail, several terms used in the context of the present invention will be defined. In addition to these terms, others are defined elsewhere in the specification as necessary. As employed above and throughout the disclosure, the following terms and abbreviations, unless otherwise indicated, shall be understood to have the following meanings. Unless otherwise expressly defined herein, terms of art used in this specification will have their art-recognized meanings.

As used in the specification and claims, the singular form “a”, “an” and “the” includes plural references unless the context clearly dictates otherwise. For example, reference to “an antibody” includes a plurality of such antibodies.

It is noted that in this disclosure, terms such as “comprises”, “comprised”, “comprising”, “contains”, “containing”, “consisting”, “consisted”, “consisting essentially of”, “includes”, “included” and the like are defined according to standard United States and international patent law practice

The term “about” is used herein to indicate that a value includes the standard deviation of error for the device or method being employed to determine the value. The term “at least about” is used herein to indicate the lower limit of a range. For example, when the term “having at least about 95% sequence identity”, it should be clear to those of skill in the art that this includes 95% sequence identity through 100% sequence identity. The use of the term “or” in the claims is used to mean “and/or” unless explicitly indicated to refer to alternatives only or the alternatives are mutually exclusive, although the disclosure supports a definition that refers to only alternatives and to “and/or.”

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, ex. hydroxyproline, γ-carboxyglutamate, and O-phosphoserine. Amino acid analogs refer to compounds that have but are not limited to the same basic chemical structure as a naturally occurring amino acid, i.e., an a carbon that is bound to a hydrogen, a carboxyl group, an amino group, and an R group, ex. homoserine, norleucine, methionine sulfoxide, methionine methyl sulfonium. Such analogs have modified R groups (ex. norleucine) or modified peptide backbones but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Macromolecular structures such as polypeptide structures may be described in terms of various levels of organization. “Primary structure” refers to the amino acid sequence of a peptide. “Secondary structure” refers to locally ordered, three dimensional structures within a polypeptide. These structures are commonly known as domains, for example enzymatic domains, extracellular domains, transmembrane domains, pore domains, or cytoplasmic tail domains. Domains are portions of a polypeptide that form a compact unit of the polypeptide. Exemplary domains include domains with enzymatic activity. A domain may be made up of sections of lesser organization such as stretches of β-sheet and α-helices. “Tertiary structure” refers to the complete three-dimensional structure of a polypeptide monomer. “Quaternary structure” refers to the three-dimensional structure formed by the noncovalent association of independent tertiary units.

The term ‘conservative amino acid substitution” indicates any amino acid substitution for a given amino acid residue, where the substitute residue is so chemically similar to that of the given residue that no substantial decrease in polypeptide function (e.g., enzymatic activity) results. Conservative amino acid substitutions are commonly known in the art and examples thereof are described, e.g., in U.S. Pat. Nos. 6,790,639, 6,774,107, 6,194167, or 5350576. In a preferred embodiment, a conservative amino acid substitution will be anyone that occurs within one of the following six groups:
Small aliphatic, substantially non-polar residues: Ala, Gly, Pro, Ser, and Thr;
Large aliphatic, non-polar residues: lie, Leu, and Val; Met;
Polar, negatively charged residues and their amides: Asp and Glu;
Amides of polar, negatively charged residues: Asn and Gln; His;
Polar, positively charged residues: Arg and Lys; His; and
Large aromatic residues: Trp and Tyr; Phe.

In a preferred embodiment, a conservative amino acid substitution will be any one of the following, which are listed as Native Residue (Conservative Substitutions) pairs: Ala (Ser); Arg (Lys); Asn (Gln; His); Asp (Glu); Gin (Asn); Glu (Asp); Gly (Pro); His (Asn; Gln); Ile (Leu; Val); Leu (Ile; Val); Lys (Arg; Gln; Glu); Met (Leu; Ile); Phe (Met; Leu; Tyr); Ser (Thr); Thr (Ser); Trp (Tyr); Tyr (Trp; Phe); and Val (lie; Leu).

The terms “polypeptide”, “oligopeptide”, “peptide” and “protein” are used interchangeably herein to refer to polymers of amino acids of any length. The polymer may be linear or branched, it may possibly comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also, included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

As used herein, an “antibody”, “antigen binding protein” and the like refers to a polypeptide comprising a region coded by an immunoglobulin gene or antibody fragments thereof that specifically binds and recognizes an antigen. An exemplary immunoglobulin (antibody) structural unit may comprise a tetramer, with each tetramer composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain and variable heavy chain refer to these light and heavy chains. Antibodies exist, for example, as intact immunoglobulins or as several well-characterized fragments produced by digestion with various peptidases. While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies or those identified using other methods known in the art

The light chains of intact antibodies, as used herein, from any vertebrate species can be assigned to one of two clearly distinct types, called kappa (κ) and lambda (λ), based on the amino acid sequences of their constant domains. All light chains contain one variable domain (VL) and one constant domain (CL) Several different types of heavy chains, as described herein, exist that define the class or isotype of an antibody. All heavy chains contain a series of immunoglobulin domains, usually with three constant domains (C_{H1}, C_{H2} and C_{H3}) and one variable domain (VH) that is important for binding antigen.

The term “variable” region comprises framework and CDRs (otherwise known as “hypervariable regions”) and refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called “Complementarity Determining Regions (CDRs)” or “hypervariable regions” both in the light chain and the heavy chain variable domains. The more highly conserved portions of variable domains are called the framework region (FR). The variable domains of native heavy and light chains each comprise multiple FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md. (1991), pages 647-669 and Chothia and Lesk J. Mol. Biol. 196:901-917 (1987)). The constant domains are not involved directly in binding an antibody to an antigen, but exhibit various effector functions, as described herein.

Properties of the four human IgG subclasses have been well established; each having distinct characteristics and engages the immune system quite differently. Human IgG subclasses are differentiated by their binding affinities for immune effector proteins including the neonatal Fc receptor (FcRn), Fc gamma receptors (FcγR), and the complement protein C1q. These receptor proteins play roles in serum half-life, antibody-dependent cell-mediated cytotoxicity (ADCC), and complement-dependent cytotoxicity (CDC), respectively. Affinity to these receptors has often been used to characterize the functional properties of antibodies (Bruggemann et al., 1987). A higher affinity to FcγR1 and FcγRIII indicate that the antibody has ADCC activity, whereas binding to the inhibitory receptor, FcγRIIb, contributes to less ADCC activity (Daeron, 1997; Armour et al., 1999; Clynes et al., 2000). Similarly, binding to C1q, the first protein in the complement cascade, indicates complement activity helping to activate phagocytes and destroy pathogens (Schifferli et al., 1986; Garred et al., 1989; Moore et al., 2010). FcRn binding is associated with antibody recycling and is correlative of in vivo half-life (Ghetie et al., 1996; Israel et al., 1996; Praetor and Hunziker, 2002; Jefferis, 2007). The unique functions of IgG subclasses assist in the design of antibody therapeutics.

In 1967, Johnson and Vaughan reported the existence of six canine immunoglobulins (Johnson and Vaughan, 1967; Johnson et al., 1967). Subsequent work narrowed the focus to IgGs for which Mazza et al. (1993) isolated four fractions from canine serum rich in IgG and separated each by gel filtration, protein A/G binding and electrophoretic mobilities. These fractions were used to obtain antibody reagents specific to canine IgGs (Mazza et al., 1994). While this work initiated a body of research investigating canine IgGs in various disease states, the functionalities of canine immunoglobulins and how they interact with immune effector proteins remained unclear. In 2001, Tang et al. (2001) provided canine IgG sequences necessary to begin to answer these questions. Like human IgGs, canine IgGs consist of four subclasses. By order of accession number [AF354264, AF354265, AF354266, and AF354267], Bergeron et al (Veterinary Immunology and Immunopathology 157 (2014) 31-41) referred to these canine IgG subclasses as A, B, C, and D, respectively. The alphabetical nomenclature associated with the canine IgG sequences is based on prevalence in the body. Bergeron et al provided functional analysis of each subclass.

Until 2014 very little was known about feline IgGs when Strietzel et al. (Veterinary Immunology and Immunopathology 158 (2014) 214-223) disclosed the functional properties associated with the two known sequences that had been previously isolated from a feline splenic cDNA library. These two IgG sequences, IgG1a and IgG1b had been isolated but not characterized (Kanai, T. H., et al., 2000 Vet Immunol, Immunopathol. 73 (1), 53-62). Strietzel et al reported a third feline IgG sequence, termed IgG2 and described the three feline IgG interactions with the identified feline FcγRI, FcγRIII, FcRn and C1q. Feline kappa and lambda light chains regions were additionally isolated.

A “functional Fc region” possesses at least one effector function of a native sequence Fc region. Exemplary “effector functions” include C1q binding; complement dependent cytotoxicity (CDC); Fc receptor binding; neonatal receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A non-limiting example of a sequence for a native Fc region sequence comprises an amino acid sequence that has between about 80-99% sequence identity to SEQ ID NO. 70 and SEQ ID NO. 72. In one embodiment the antibody of the invention comprises a native Fc region comprising SEQ ID No. 70. In one embodiment the antibody of the invention comprises a native Fc region comprising SEQ ID NO. 72. The native Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith. A “variant Fc region” or a “mutated” or “mutant” Fc region comprises an amino acid sequence which differs from that of a native sequence Fc region by virtue of at least one amino acid modification and may or may not retain at least one effector function of the native sequence Fc region as compared to the native Fc region sequence. Preferably, the variant Fc region has at least one amino acid substitution compared to a native sequence Fc region or to the Fc region of a parent polypeptide, ex. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith. A variant or mutated Fc region may also essentially eliminate the function of the Fc region of the antibody. A variant or mutated Fc region may also add or enhance the function of the Fc region of an antibody. For example, Fc region mutations may eliminate effector function of an antibody. In another example a mutated Fc region may enhance effector function of an antibody. In yet another example, a mutated Fc region may alter the half-life or affect the binding of other factors in a cell that may determine properties of the antibody. In one embodiment the antibody of the invention comprises a mutated Fc region. In one embodiment the antibody of the invention comprises a variant or mutated Fc region that affects effector function comprising an amino acid sequence comprising between about 80-99% sequence identity to SEQ ID NO. 70. In one embodiment the antibody of the invention comprises a variant or mutated Fc region that affects effector function comprising the amino acid sequence comprising between about 80-99% sequence identity to SEQ ID NO. 72. In one embodiment the antibody of the invention comprises a variant or mutated Fc region that affects effector function comprising the amino acid sequence comprising SEQ ID NO. 70. In one embodiment the antibody of the invention comprises a variant or mutated Fc region that affects effector function comprising the amino acid sequence comprising SEQ ID NO. 72.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, for example, in an animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202: 163 (1996), may be performed.

For preparation of antibodies, ex. recombinant, monoclonal, or polyclonal antibodies, many techniques known in the art may be used. The genes encoding the heavy and light chains of an antibody of interest can be cloned from a cell and used to produce a recombinant monoclonal antibody. Gene libraries encoding heavy and light chains of monoclonal antibodies may also be used. Random combinations of the heavy and light chain gene products generate a large pool of antibodies with different antigenic specificity. Techniques to produce single chain antibodies or recombinant antibodies are found in the art and may be adapted to produce antibodies to polypeptides according to the invention. Phage display technology may also be used to identify antibodies and heteromeric fragments that specifically bind to selected antigens. Antibodies may also be made bispecific, i.e., able to recognize two different antigens, or heteroconjugates, ex. two covalently joined antibodies, or immunotoxins.

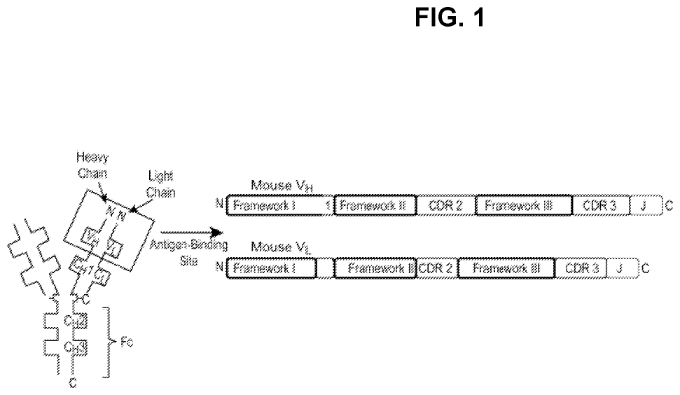

“Native antibodies” and “native immunoglobulins” are usually heterotetrameric glycoproteins of about 150,000 Daltons, composed of two identical light (I) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. FIG. 1 is an example of the general structure of a native mouse immunoglobulin G (IgG) highlighting the antigen binding site.

As used herein, the term “antigen binding protein”, “antibody”, “antagonist antibody”, “antigen binding fragment” and the like, which may be used interchangeably herein, refers to a polypeptide, or fragment thereof, comprising an antigen binding site. Thus, an isolated antibody or fragment may be a polyclonal antibody, a monoclonal antibody, a synthetic antibody, a recombinant antibody, a chimeric antibody, a heterochimeric antibody, a caninized antibody, a felinized antibody, a fully canine antibody or a fully feline antibody.

In some embodiments, the term “antigen binding protein” “antibody” “antagonist antibody” and the like preferably refers to monoclonal antibodies and fragments thereof, and immunologic binding equivalents thereof that can bind to a TGFβ protein and fragments thereof. Exemplary antibody fragments include Fab, Fab′, F(ab′)₂, Fv, scFv, Fd, dAb, diabodies, their antigen-recognizing fragments, small modular immunopharmaceuticals (SMIPs) nanobodies, IgNAR molecules and the equivalents that are recognized by one of skill in the art to be an antibody or antibody fragment and any of above mentioned fragments and their chemically or genetically manipulated counterparts, as well as other antibody fragments and mutants thereof, fusion proteins comprising an antibody portion, and any other modified configuration of the immunoglobulin molecule that comprises an antigen recognition site. Antibodies and antigen binding proteins can be made, for example but not limited to, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)) or other techniques employed and well known by those of skill in the art.

A “monoclonal antibody” as defined herein is a single pure homogeneous type of antibody. All monoclonal antibodies produced are identical and have the same antigen specificity. Monoclonal antibodies are a homogeneous antibody population wherein the monoclonal antibody is comprised of amino acids (naturally occurring and non-naturally occurring) that are involved in the selective binding of an antigen. A population of monoclonal antibodies is highly specific, being directed against a single antigenic site. The term “monoclonal antibody” encompasses not only intact monoclonal antibodies and full-length monoclonal antibodies, but also fragments thereof (Fab, Fab′, F(ab′)₂, Fv, scFv, Fd, dAb, diabodies, their antigen-recognizing fragments, small modular immunopharmaceuticals (SMIPs) nanobodies, IgNAR molecules and the like), mutants thereof, fusion proteins comprising an antibody portion, and any other modified configuration of the immunoglobulin molecule that comprises an antigen recognition site of the required specificity and the ability to bind to an antigen. It is not intended to be limited to the source of the antibody or the manner in which it is made (ex. by hybridoma, phage selection, recombinant expression, transgenic animals, etc.).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-recognition and -binding site. This region consists of a dimer of one heavy chain and one light chain variable domain in tight, noncovalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

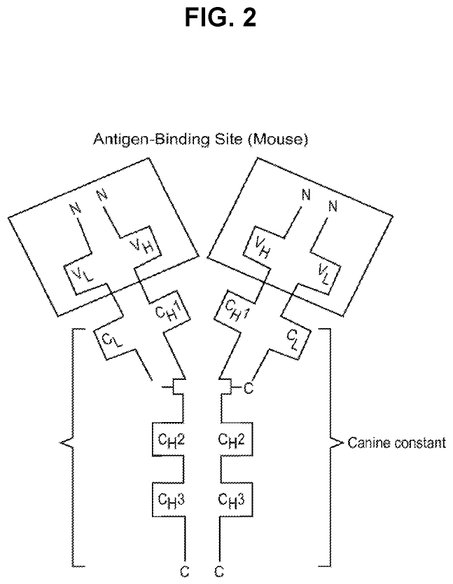

The monoclonal antibodies described herein specifically include “chimeric” antibodies (immunoglobulins) in which a portion of the heavy and/or light chain is identical with or homologous to corresponding sequences in antibodies derived from a particular species, while the remainder of the chain(s) is identical with or homologous to corresponding sequences in antibodies derived from another species, as well as fragments of such antibodies, so long as they exhibit the desired biological activity. Typically, chimeric antibodies are antibodies whose light and heavy chain genes have been constructed, typically by genetic engineering, from antibody variable and constant region genes belonging to different species. For example, the variable segments of the genes from a mouse monoclonal antibody (or any other species antibody including feline) may be joined to canine constant segments, for example the amino acid sequence of the IgGB canine heavy chain constant region represented herein by SEQ ID NO. 72 both with and without effector function mutations or IgG1a feline heavy chain constant region represented herein by SEQ ID NO. 70 both with and without effector function mutations. Additionally, a chimeric feline antibody is produced in the same fashion except that the amino acid sequence comprising SEQ ID NO. 75, the feline heavy chain constant region, is joined to the variable segments of another species antibody (mouse, canine, feline etc.). FIG. 2 is a schematic representation of the general structure of one embodiment of a mouse: canine IgG. In this embodiment the antigen binding site is derived from mouse while the Fc portion is canine. This illustration does not limit the claimed invention solely to a mouse/canine chimera but can also be applied to combinations of any species antibodies: canine, feline, murine and human to list a few, as described herein.

The term “heterochimeric” as defined herein, refers to an antibody in which one of the antibody chains (heavy or light) is speciated (i.e. caninized or felinized) while the other is chimeric. In this embodiment, a caninized variable heavy chain (where all of the CDRs are mouse and all FRs are canine) is paired with a chimeric variable light chain (where all of the CDRs are mouse and all FRs are mouse. In this embodiment, both the variable heavy and variable light chains are fused to a canine constant region. As with the chimeric antibodies, there are no limitations on the combinations of species and portions of antibodies.

The term “canine antibody”, “feline antibody”, “human antibody” and the like, as used herein, refers to an antibody that is generated against a target and antibodies isolated from lymphocytes from within the target species. These antibodies, as described herein, have been recombinantly modified in vitro to include specific constant regions of the target species or otherwise recombinantly modified.

The phrase “recombinant canine antibody”, “recombinant feline antibody”, “recombinant human antibody” and the like all include speciated antibodies that are prepared, expressed, created or isolated by recombinant means, such as antibodies expressed using a recombinant expression vector transfected into a host cell, antibodies isolated from a recombinant, combinatorial canine (or feline, human, etc.) antibody library, antibodies isolated from an animal (ex. a mouse) that is transgenic for canine, feline or other species immunoglobulin genes (see ex. Taylor, L. D., et al. (1992) Nucl. Acids Res. 20:6287-6295) or antibodies prepared, expressed, created or isolated by any other means that involves recombining canine (or feline, human etc.) immunoglobulin gene sequences to other DNA sequences.

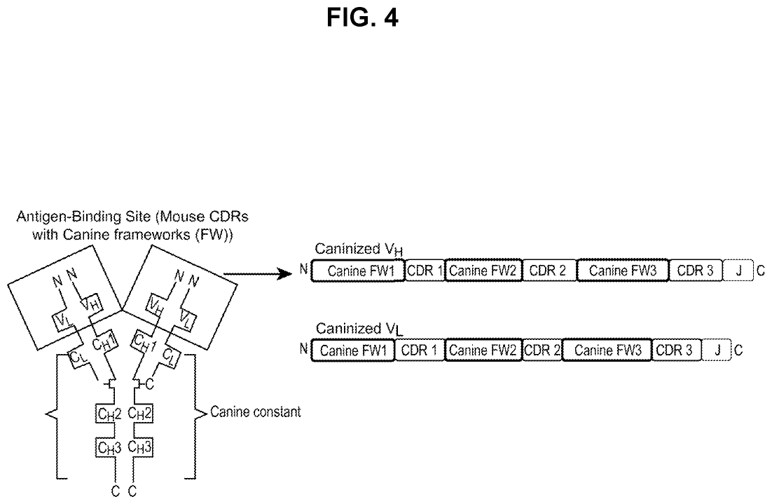

For the sake of simplicity, the following describes “caninized” antibodies, however the same can be applied to felinized, humanized or any other speciated antibody. As an example, “caninization” is defined as a method for transferring non-canine antigen-binding regions from a donor antibody to a less immunogenic canine antibody acceptor to generate treatments useful as therapeutics in dogs. Caninized antibodies are canine antibody sequences in which hypervariable region residues of the recipient are replaced by hypervariable region residues from a non-canine species (donor antibody) such as such as mouse, rat, rabbit, cat, dogs, goat, chicken, bovine, horse, llama, camel, dromedaries, sharks, non-human primates, human, humanized, recombinant sequence, or an engineered sequence having the desired properties, specificity, affinity, and capacity. Furthermore, caninized antibodies may include residues that are not found in the recipient antibody or in the donor antibody. These modifications are made to further refine antibody performance. The modifications to the hypervariable regions and/or the framework regions, as described herein, are determined for each separately engineered speciated (caninized) antibody based on experimentation known to those in the art yet cannot be predicted prior to said experimentation. The caninized antibody optionally may comprise a complete, or at least a portion of an immunoglobulin constant region (Fc), typically that of a canine immunoglobulin. FIG. 4 is an illustration of one embodiment showing speciation or caninization of a mouse IgG. In this embodiment, mouse CDRs are grafted onto canine frameworks. In some cases, mouse frameworks or residues therein that are outside of the hypervariable region are maintained. All descriptions of caninization of an antibody and that of a caninized antibody can be applicable, in concept, to any “speciated” antibody, whether it is caninization, felinization, humanization etc.

The “parent” antibody, as described herein, is one that is encoded by an amino acid sequence used for the preparation of the variant. Preferably, with caninized or canine antibodies the parent antibody has a canine framework region and, if present, has canine antibody constant region(s). For example, the parent antibody may be a caninized or canine antibody. The same is true for felinized, humanized, equinized, bovinized antibodies.

The term “backmutation” refers to a process in which some or all of the somatically mutated amino acids of a canine antibody are replaced with the corresponding germline residues from a homologous germline antibody sequence. The heavy and light chain sequences of the canine antibody of the invention are aligned separately with the germline sequences to identify the sequences with the highest homology. Differences in the canine antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the canine antibody should not be included in the final canine antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and co-linear to the sequence of the canine antibody of the invention. Back mutation of selected target framework residues to the corresponding donor residues might be required to restore and or improved affinity.

An “antigen” is a molecule, or a portion of a molecule, capable of being bound by an antibody. In general, epitopes consist of chemically active surface groupings of molecules, for example, amino acids or sugar side chains, and have specific three-dimensional structural characteristics as well as specific charge characteristics. Epitopes are the antigenic determinant on a protein that is recognized by the immune system. The components of the immune system recognizing epitopes are antibodies, T-cells, and B-cells. T-cell epitopes are displayed on the surface of antigen-presenting cells (APCs) and are typically 8-11 (MHC class I) or 15 plus (MHC class II) amino acids in length. Recognition of the displayed MHC-peptide complex by T-cells is critical to their activation. These mechanisms allow for the appropriate recognition of “self”-versus “non-self” proteins such as bacteria and viruses. Independent amino acid residues that are not necessarily contiguous contribute to interactions with the APC binding cleft and subsequent recognition by the T-Cell receptor (Janeway, Travers, Walport, Immunobiology: The Immune System in Health and Disease. 5^{th} edition New York: Garland Science; 2001). Epitopes that are recognized by soluble antibodies and cell surface associated B-cell receptors vary greatly in length and degree of continuity (Sivalingam and Shepherd, Immunol. 2012; 51(3-4): 304-309). Again, even linear epitopes or epitopes found in a continuous stretch of protein sequence will often have discontiguous amino acids that represent the key points of contact with the antibody paratopes or B-cell receptor. Epitopes recognized by antibodies and B-cells can be conformational with amino acids comprising a common area of contact on the protein in three-dimensional space and are dependent on tertiary and quaternary structural features of the protein. These residues are often found in spatially distinct areas of the primary amino acid sequence.

As used herein, the term “TGF beta”, “TGF p” and “TGFB”, as used interchangeably herein, refers to Transforming Growth Factor Beta protein 1 (TGFβ1), Transforming Growth Factor Beta protein 2 (TGFβ2) and Transforming Growth Factor Beta protein 3 (TGFβ3). TGFβ proteins are part of a superfamily of related growth factors that exert pleiotropic effects on wound healing by regulating cell proliferation and migration, cellular differentiation, apoptosis, ECM (extra cellular matrix) production and immune modulation. As used herein, the inhibition of TGFβ proteins through use of the antibodies of the invention are used to treat TGFβ related disorders such as fibrosis disorders, bone disorders and cell proliferation disorders.

As used herein, an “anti-TGFβ antibody” can be interchangeably termed “anti-TGFβ antigen binding protein” and “anti-TGFβ antagonist antibody”, “anti-TGFβ antigen binding fragment”, “anti-TGFβ antigen binding portion” and the like describing any functional molecule that inhibits binding of TGFβ1, TGFβ2 and TGFβ3 proteins from binding to its specific receptor thus inhibiting the biological function of the TGFβ signaling pathways associated thereof. In a preferred embodiment, the anti-TGFβ antibody binds to the TGFβ1, 2 and 3 proteins. The anti-TGFβ antibody of the invention encompass binding proteins and antibodies that block, antagonize, suppress or reduce (including significantly reduce) TGFβ biological activity, including downstream pathways mediated by TGFβ1, TGFβ2 and TGFβ3 signaling, and/or inhibit TGFβ proteins from binding the TGFR2 receptor, such as receptor binding and/or elicitation of a cellular response to TGFβ1, TGFβ2 and TGFβ3 proteins. For purpose of the present invention, it will be explicitly understood that the term “anti-TGFβ antibody” or “anti-TGFβ-antagonist antibody” or “TGFβ B antigen binding protein” encompass all the previously identified terms, titles, and functional states and characteristics whereby the biological activity of TGFβ itself including, but not limited to, its ability to mediate any aspect of the development or treatment of a TGFβ related disorder such as fibrosis disorder, bone disorders and/or cell proliferation disorders or the consequences of the biological activity, are substantially nullified, decreased, or neutralized to any meaningful degree. Examples of anti-TGFβ antibodies are provided herein.

A “variant” anti-TGFβ antibody, refers herein to a molecule which differs in amino acid sequence from a “parent” anti-TGFβ antibody amino acid sequence by virtue of addition, deletion, and/or substitution of one or more amino acid residue(s) in the parent antibody sequence and retains at least one desired activity of the parent anti-TGFβ-antibody. Desired activities can include the ability to bind the antigen specifically, the ability to reduce, inhibit or neutralize TGFβ activity in an animal, and the ability to inhibit TGFβ-mediated SMAD signaling in a cell-based assay. In one embodiment, the variant comprises one or more amino acid substitution(s) in one or more hypervariable and/or framework region(s) of the parent antibody. For example, the variant may comprise at least one, or from about one to about ten or from about two to about five, substitutions in one or more hypervariable and/or framework regions of the parent antibody. Ordinarily, the variant will have an amino acid sequence having at least 50% amino acid sequence identity with the parent antibody heavy or light chain variable domain sequences or at least between about 65%, 75%, 80%, 85%, 90%, 91%, 92%, 93%, 94% 95%, 96%, 97%, 98% or 99% sequence identity with the parent antibody. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting overall sequence identity or homology. The variant retains the ability to bind an TGFβ variant may have a stronger binding affinity, enhanced ability to reduce, inhibit or neutralize TGFβ activity in an animal, and/or enhanced ability to inhibit TGFβ-mediated SMAD signaling in a cell-based assay.

“TGFβ receptor” refers to a polypeptide that is bound by or activated by a TGFβ protein. TGFβ receptors are single-pass serine/threonine kinase receptors that belong to TGFβ receptor family. They exist in several different isoforms that can be homo- or heterodimeric. Three TGFβ receptors specific for TGFβ proteins can be distinguished by their structural and functional properties. TGFβ R1 (ALK5) and TGFβ R2 have similar ligand-binding affinities. Both TGFβ R1 and TGFβ R2 have a high affinity for TGFβ1 and low affinity for TGFβ2. TGFβR3 (p-glycan) has a high affinity for both homodimeric TGFβ1 and TGFβ2 and in addition the heterodimer TGFβ1,2. The TGFβ receptors also bind TGFβ3. Mechanistically TGFβ proteins initially bind to TGFβR2 receptor, which recruits and phosphorylates TGFβR1. TGFβR1 then phosphorylates receptor-regulated SMADs (R-SMADs) which can then bind the co-SMAD SMAD4. R-SMAD/co-SMAD complexes accumulate in the nucleus where they act as transcription factors and participate in the regulation of target gene expression

The term “neutralize” as used herein with respect to an activity of a monoclonal antibody of the invention means the ability to substantially antagonize, prohibit, prevent, restrain, slow, disrupt, eliminate, stop, reduce or reverse progression or severity of that which is being inhibited including, but not limited to, a biological activity or property, a disease or a condition. The inhibition or neutralization is preferably at least about 10%, 20%, 30%, 40%, 50%, 60%, 70%, 80%, 90%, 95% or higher. An antibody is said to “neutralize” its antigen if antibody binding to the antigen results in partial or complete inhibition or reduction of a biological function of the antigen. Neutralization of a TGFβ protein's biological activity is assessed by measuring the partial or complete inhibition or reduction of one or more in vitro or in vivo indicators of TGFβ activity such as, differences in TGFβ receptor binding and signaling pathways. The ability to neutralize TGFβ activity is assessed, as described herein, by measuring the inhibition of Smad2 phosphorylation, as described in the in vitro assays described herein. The neutralization of TGFβ in vivo may result in inhibition of cell phenotype switching, cell proliferation, and cell survival due to TGFβ in conditions of disease.

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins, additionally also meaning that the antibody of the invention will also bind the target antigen at the epitope in vivo). An epitope that “specifically binds”, or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance comprising said antigen than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a TGFβ epitope is a protein that binds this epitope with greater affinity, avidity, more readily, and/or with greater duration than it binds to other epitopes or non-TGFB epitopes.

The term “specifically” in the context of antibody binding, refers to high avidity and/or high affinity binding of an antibody to a specific antigen, i.e., a polypeptide, or epitope. Antibody specifically binding an antigen is stronger than binding of the same antibody to other antigens. Antibodies which bind specifically to a polypeptide may be capable of binding other polypeptides at a weak, yet detectable level (for example, 10% or less of the binding shown to the polypeptide of interest). Such weak binding, or background binding, is readily discernible from the specific antibody binding to a subject polypeptide, e.g. by use of appropriate controls. In general, specific antibodies bind to an antigen with a binding affinity with a K_{D} of 10⁻⁷ M or less, 10⁻⁸ M or less 10⁻⁹ M or less, 10⁻¹⁰ M or less, 10⁻¹¹ M or less, 10⁻¹² M or less, or 10⁻¹³ M or less etc.

As used herein, the term “affinity” refers to the strength of the binding of a single antigen-combining site with an antigenic determinant. Affinity depends on the closeness of stereochemical fit between antibody and antigen determinants, on the size of the area of contact between them, on the distribution of charged and hydrophobic groups, etc. Antibody affinity can be measured by equilibrium analysis or by the Surface Plasmon Resonance “SPR” method (for example BIACORE™) The SPR method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The term “K_{D}”, as used herein, is intended to refer to the dissociation constant of an antibody-antigen interaction. The dissociation constant, K_{D}, and the association constant, Kₐ, are quantitative measures of affinity. At equilibrium, free antigen (Ag) and free antibody (Ab) are in equilibrium with antigen-antibody complex (Ag-Ab), and the rate constants, kₐ and k_{d}, quantitate the rates of the individual reactions. At equilibrium, kₐ [Ab][Ag]=k_{d} [Ag−Ab]. The dissociation constant, K_{d}, is given by: K_{D}=k_{d}/kₐ=[Ag][Ab]/[Ag−Ab]. K_{D} has units of concentration, most typically M, mM, μM, nM, pM, etc. When comparing antibody affinities expressed as K_{D}, having greater affinity for TGFβ is indicated by a lower value. The association constant, Kₐ, is given by: Kₐ=kₐ/k_{d}=[Ag−Ab]/[Ag][Ab]. Kₐ has units of inverse concentration, most typically M⁻¹, mM⁻¹, μ·M⁻¹, nM⁻¹, pM⁻¹, etc. As used herein, the term “avidity” refers to the strength of the antigen-antibody bond after formation of reversible complexes. Anti-TGFβ antibodies may be characterized in terms of the K_{D} for their binding to a TGFβ protein, as binding “with a dissociation constant (K_{D}) in the range of from about (lower K_{D} value) to about (upper K_{D} value).”

The terms “nucleic acid”, “polynucleotide”, “nucleic acid molecule” and the like may be used interchangeably herein and refer to a series of nucleotide bases (also called “nucleotides”) in DNA and RNA. The nucleic acid may contain deoxyribonucleotides, ribonucleotides, and/or their analogs. The term “nucleic acid” includes, for example, single-stranded and double-stranded molecules. A nucleic acid can be, for example, a gene or gene fragment, exons, introns, a DNA molecule (ex. cDNA), an RNA molecule (ex. mRNA), recombinant nucleic acids, plasmids, and other vectors, primers and probes. Both 5′ to 3′ (sense) and 3′ to 5′ (antisense) polynucleotides are included. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (for example, methyl phosphonates, phosphotriesters, phosphoamidates, cabamates, etc.) and with charged linkages (ex. phosphorothioates, phosphorodithioates, etc.), those containing pendant moieties, such as, for example, proteins (ex. nucleases, toxins, antibodies, signal peptides, poly-L-lysine, etc.), those with intercalators (ex. acridine, psoralen, etc.), those containing chelators (ex., metals, radioactive metals, boron, oxidative metals, etc.), those containing alkylators, those with modified linkages (ex. alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. Vectors, as described herein, have expression control sequences meaning that a nucleic acid sequence that directs transcription of a nucleic acid. An expression control sequence can be a promoter, such as a constitutive or an inducible promoter, or an enhancer. The expression control sequence is ‘operably linked’ to the nucleic acid sequence to be transcribed. A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Just as a polypeptide may contain conservative amino acid substitution(s), a polynucleotide thereof may contain conservative codon substitution(s). A codon substitution is considered conservative if, when expressed, it produces a conservative amino acid substitution, as described above. Degenerate codon substitution, which results in no amino acid substitution, may also be useful in polynucleotides of the present invention. Thus, for example, a polynucleotide encoding a selected polypeptide useful in an embodiment of the present invention may be mutated by degenerate codon substitution in order to approximate the codon usage frequency exhibited by an expression host cell to be transformed therewith, or to otherwise improve the expression thereof.

A “variant” nucleic acid refers herein to a molecule which differs in sequence from a “parent” nucleic acid. Polynucleotide sequence divergence may result from mutational changes such as deletions, substitutions, or additions of one or more nucleotides. Each of these changes may occur alone or in combination, one or more times in a given sequence.

The term “isolated” means that the material (for example, antibody as described herein or nucleic acid) is separated and/or recovered from a component of its natural environment. Contaminant components of its natural environment are materials that would interfere with diagnostic or therapeutic uses for the material, and may include enzymes, hormones, and other proteinaceous or non-proteinaceous solutes. With respect to nucleic acid, an isolated nucleic acid may include one that is separated from the 5′ to 3′ sequences with which it is normally associated in the chromosome. In preferred embodiments, the material will be purified to greater than 95% by weight of the material, and most preferably more than 99% by weight. Isolated material includes the material in situ within recombinant cells since at least one component of the material's natural environment will not be present. Ordinarily, however, isolated material will be prepared by at least one purification steps used herein.

The terms “cell”, “cell line”, and “cell culture” may be used interchangeably. These terms also include their progeny, which are all subsequent generations. It is understood that all progeny may not be identical due to deliberate or inadvertent mutations. In the context of expressing a heterologous nucleic acid sequence, “host cell” refers to a prokaryotic or eukaryotic cell (for example, bacterial cells, yeast cells, mammalian cells, and insect cells) whether located in vitro or in vivo. For example, host cells may be located in a transgenic animal. Host cell can be used as a recipient for vectors and may include any transformable organism that is capable of replicating a vector and/or expressing a heterologous nucleic acid encoded by a vector.

The word “label” when used herein refers to a detectable compound or composition that is conjugated directly or indirectly to the antibody or nucleic acid. The label may itself be detectable by itself (for example, radioisotope labels or fluorescent labels) or, in the case of an enzymatic label, may catalyze chemical alteration of a substrate compound or composition that is detectable.

A “subject” or “patient” refers to an animal in need of treatment that can be affected by molecules of the invention. Animals that can be treated in accordance with the invention include vertebrates, specifically mammals such as a canine or feline being particularly preferred examples.

A “composition” is intended to mean a combination of active agent, whether chemical composition, biological composition or biotherapeutic (particularly antibodies as described herein) and another compound or composition which can be inert (for example, a label), or active, such as an adjuvant.

As defined herein, “pharmaceutically acceptable carriers” suitable for use in the invention are well known to those of skill in the art. Such carriers include but are not limited to, water, saline, buffered saline, phosphate buffer, alcohol/aqueous solutions, emulsions or suspensions. Other conventionally employed diluents, adjuvants and excipients, may be added in accordance with conventional techniques. Such carriers can include ethanol, polyols, and suitable mixtures thereof, vegetable oils, and injectable organic esters. Buffers and pH adjusting agents may also be employed. Buffers include, without limitation, salts prepared from an organic acid or base. Representative buffers include, without limitation, organic acid salts, such as salts of citric acid, citrates, ascorbic acid, gluconic acid, histidine-Hel, carbonic acid, tartaric acid, succinic acid, acetic acid, or phthalic acid, Tris, trimethanmine hydrochloride, or phosphate buffers. Parenteral carriers can include sodium chloride solution, Ringer's dextrose, dextrose, trehalose, sucrose, and sodium chloride, lactated Ringer's or fixed oils. Intravenous carriers can include fluid and nutrient replenishers, electrolyte replenishers, such as those based on Ringer's dextrose and the like. Preservatives and other additives such as, for example, antimicrobials, antioxidants, chelating agents (ex. EDTA), inert gases and the like may also be provided in the pharmaceutical carriers. The present invention is not limited by the selection of the carrier. The preparation of these pharmaceutically acceptable compositions, from the above-described components, having appropriate pH isotonicity, stability and other conventional characteristics is within the skill of the art. See, for example, texts such as Remington: The Science and Practice of Pharmacy, 20th ed, Lippincott Williams & Wilkins, publ., 2000; and The Handbook of Pharmaceutical Excipients, 4.sup.th edit., eds. R. C. Rowe et al, APhA Publications, 2003.

A “therapeutically effective amount” (or “effective amount”) refers to an amount of an active ingredient, for example, an agent according to the invention, sufficient to effect beneficial or desired results when administered to a subject or patient. An effective amount can be administered in one or more administrations, applications or dosages. A therapeutically effective amount of a composition according to the invention may be readily determined by one of ordinary skill in the art. In the context of this invention, a “therapeutically effective amount” is one that produces an objectively measured change in one or more parameters associated TGFB related condition(s) sufficient to effect beneficial or desired results including clinical results such as alleviation or reduction in pain sensation. An effective amount can be administered in one or more administrations. For purposes of this invention, an effective amount of composition is an amount sufficient to prevent, treat, reduce or eliminate a TGFβ related disorder, which is defined herein as a fibrosis disorder, a bone disorder or a cell proliferation disorder. The therapeutically effective amount will vary depending upon the particular subject and condition being treated, the weight and age of the subject, the severity of the condition, the particular composition chosen, the dosing regimen to be followed, timing of administration, the manner of administration and the like, all of which can readily be determined by one of ordinary skill in the art.

As used herein, the term “therapeutic” encompasses the full spectrum of treatments for a disease, condition or disorder. A “therapeutic” agent of the invention may act in a manner that is prophylactic or preventive, including those that incorporate procedures designed to target subjects that can be identified as being at risk; or in a manner that is ameliorative or curative in nature; or may act to slow the rate or extent of the progression of at least one symptom of a disease or disorder being treated.

In a further aspect, the invention features veterinary compositions in which antibodies of the present invention are provided for therapeutic or prophylactic uses. The invention features a method for treating a canine or feline subject having a particular antigen, for example, one associated with a disease or condition. The method includes administering a therapeutically effective amount of an antibody specific for one or more TGFβ proteins with the antibody of the invention as described herein.

The antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate.

A composition comprising the antibody of the invention may be administered to a subject exhibiting pathologies or disorders as described herein using standard administration techniques including intravenous, intraperitoneal, subcutaneous, pulmonary, transdermal, intramuscular, intranasal, buccal, sublingual, or suppository administration. The route of administration of an antibody of the invention may be parenteral. Infusions typically are given by intravenous route. Preferably, antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Further, the subject of the method of the invention is also referred to as the patient and is described herein as a canine or feline.

A TGFβ related disorder, as used herein, is a disorder in which the regulation or overall levels of one or more TGFβ proteins leads to a connective tissue disorder, a fibrosis/fibrotic disorder, a bone disorder or a cell proliferation disorder. TGFβ regulates diverse cellular functions including proliferation, apoptosis, differentiation and inflammation and as such a dysregulation of these proteins can lead to several of the named disorders.

As used herein a connective tissue disorder refers to a group of disorders involving the protein-rich tissue that supports organs and other parts of the body. Examples of connective tissue are fat, bone, and cartilage. These disorders often involve the joints, muscles, and skin, but they can also involve other organs and organ systems, including the eyes, heart, lungs, kidneys, gastrointestinal tract, and blood vessels.

Fibrosis related disorders, as described herein, relates to a pathologic process which includes scar formation and over production of extracellular matrix by the connective tissue as a response to tissue damage. The molecular process is not different from normal formation of connective tissue and extracellular matrix in the normal organs. Physiologically, fibrosis acts to deposit connective tissue, which can interfere with or completely inhibit the normal architecture and function of the underlying organ or tissue. Fibrosis can be used to describe the pathological state of excess deposition of fibrous tissue, as well as the process of connective tissue deposition in healing. Defined by the pathological accumulation of extracellular matrix (ECM) proteins, fibrosis results in scarring and thickening of the affected tissue, it is in essence an exaggerated wound healing response which interferes with normal organ function. Fibrosis formation includes interaction between many cell types and cytokines, and when the balance becomes profibrotic, there is fibrosis formation. Fibrosis is similar to the process of scarring, in that both involve stimulated fibroblasts laying down connective tissue, including collagen and glycosaminoglycans. The process is initiated when immune cells such as macrophages release soluble factors that stimulate fibroblasts. The most well characterized pro-fibrotic mediator is TGFβ which is released by macrophages as well as any damaged tissue between surfaces called interstitium. Fibrotic conditions, as defined herein, are selected from the group consisting of: pulmonary fibrosis which includes both cystic and idiopathic pulmonary fibrosis; cirrhosis of the liver; glial scarring in the brain, arthrofibrosis in the knee, shoulder and other joints, retroperitoneal fibrosis, systemic sclerosis (scleroderma), and in particular kidney fibrosis leading to chronic kidney disease (CKD). Fibrosis is a progressive degenerative disorder of the blood vessels, skin, lungs, kidneys, heart and GI tract and until the present is considered an irreversible process and has classically been treated by anti-inflammatory and immunosuppressive agents, which many times causes harm.

Chronic Kidney Disease (CKD), as described herein, involves a loss of functional kidney tissue due to a prolonged, progressive fibrotic process. Dramatic changes in kidney structure may be seen, although structural and functional changes in the kidney are only loosely correlated. Disease is usually present for many months or years before it becomes clinically apparent, and it is invariably irreversible. Many causes of CKD are associated with progressive interstitial fibrosis. The severity of interstitial fibrosis is positively correlated to the magnitude of decline in GFR and negatively correlated with the prognosis. The glomerular, tubulointerstitial, and vascular lesions found in animals with generalized CKD are often similar, regardless of the initiating cause. TGFβ has been described as the most important pro-fibrotic mediator responsible for myofibroblast activation. It drives a convergent pathway that integrates the effects of many other fibrogenic factors. TGFβ1 is the most abundant isoform and is synthesized by all cell types of the kidney. TGFβ, as well as functioning as a profibrotic cytokine as discussed, is also an abundant bone matrix protein that influences the formation, function and cell-cell interactions of osteoblasts and osteoclasts to control bone remodeling and maintain adequate bone mass and it has been shown that TGFβ inhibition is a potential mechanism for decreasing bone demineralization during secondary renal hyperparathyroidism (SRHP) due to CKD.

“Treatment” “treating”, and the like refers to both therapeutic treatment and prophylactic or preventative measures. Animals in need of treatment include those already with the disorder as well as those in which the disorder is to be prevented from starting or progressing. Treatment may also be described as delaying the onset or delaying the severity of the onset of symptoms or condition. The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

Before the present methods are described, it is to be understood that this invention is not limited to particular methods, and experimental conditions described, as such methods and conditions may vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

The invention disclosed herein concerns antibodies (used interchangeably with the terms “antigen binding proteins”, “antagonist antibodies” “antibody fragments” and the like, as described herein), that specifically bind to TGFβ1, TGFβ2 and TGFβ3 proteins and in particular antibodies, whether it be canines or felines, caninized or felinized produced by recombinant methods, hybridoma technologies or phage display technology or fully speciated monoclonal antibodies that specifically binds to canine or feline TGFβ1, TGFβ2 and TGFβ3 thus preventing all from binding to the TGFβRII receptors, thus serving as an antagonist in that the signaling pathway is prevented from being activated by one of the TGFβ proteins. In a preferred embodiment the present invention provides an antibody that binds to TGFβ1 and TGFβ2 and TGFβ3.

While the properties of antibodies make them very attractive therapeutic agents, there are a number of limitations. The vast majority of monoclonal antibodies (mAbs) are of rodent origin, as previously noted. When such antibodies are administered in a different species, patients/subjects can mount their own antibody response to such xenogenic antibodies. Such response may result in the eventual neutralization and elimination of the antibody. As described above mice are used extensively in the production of monoclonal antibodies, although production of antibodies is not limited to mice. Species such as canines can be immunized with an antigen and antibodies recovered and characterized One problem in the using of antibodies produced by a particular species, for example if originally generated in a mouse, is that a non-murine subject being treated with said antibodies react to the mouse antibodies as if they were a foreign substance thus creating a new set of antibodies to the mouse antibodies. Mouse antibodies are “seen” by the non-murine, for example, the canine (or any other non-murine species), immune system will “see” a xenogenic antibody as foreign and may then mount an immune response against the molecule. Those skilled in the field will recognize the need to be able to treat a subject with an antigen specific antibody but have that antibody species specific for use. Part of the reaction generated from cross species antibody administration, for example a mouse monoclonal antibody being administered to a canine, can range from a mild form, like a rash, to a more extreme and life-threatening response, such as renal failure. This immune response can also decrease the effectiveness of the treatment or create a future reaction if the subject is given a subsequent treatment containing mouse antibodies. Accordingly, as set forth in the present invention set forth to overcome this disadvantage by “caninization” or “felinizing” of the antibody of the invention. In particular this process focuses on the framework regions of the immunoglobulin variable domain but could also include the complementarity determinant regions (CDR's) of the variable domain. The enabling steps and reduction to practice for this process are described in this disclosure and the process of affinity maturation around the sequences of the CDRs is well known in the field.

The process of modifying a monoclonal antibody (antibody, antagonist antibody etc as described herein and terms used interchangeably) from an animal to render it less immunogenic for therapeutic administration to a different species has been aggressively pursued and has been described in a number of publications (e.g. Antibody Engineering: A practical Guide. Carl A. K. Borrebaeck ed. W.H. Freeman and Company, 1992). However, this process has not been routinely applied for the development of therapeutic or diagnostics for non-humans until recently. In fact, very little has been published with regard to canine, feline, or other species-specific variable domains at all. Wasserman and Capra, Biochem. 6, 3160 (1977), determined the amino acid sequence of the variable regions of both a canine heavy chain. Wasserman and Capra, Immunochem. 15, 303 (1978), determined the amino acid sequence of the K light chain from a canine IgA. McCumber and Capra, Mol. Immunol. 16, 565 (1979), disclose the complete amino-acid sequence of a canine mu chain. Tang et al., Vet. Immunology Immunopathology 80, 259 (2001), discloses a single canine IgG-A y chain cDNA and four canine IgG-A y chain protein sequences. Bergeron et al. describes the functional properties of the four canine heavy chains. To this point the paucity of information available on canine antibodies has prevented their development as therapeutics for the treatment canine disease.

These noted limitations have prompted the development of engineering technologies known as “speciation” that is well known to those in the art in terms of “humanization” of therapeutic antibodies. The “caninization”, “felinization”, “humanization” of antibodies are a few examples of the technique of “speciation”, These molecules are generated as antibodies or fragments which contain minimal sequence derived from non-target immunoglobulin. As an example, caninized antibodies (“target species antibody”) in which residues from a complementarity determining region (CDR) of the recipient/target are replaced by residues from a CDR of a non-target species (i.e. “donor antibody” or “originating species antibody”) such as mouse, having the desired properties such as specificity, affinity, and potency. This strategy is based on identifying the most appropriate target (germline antibody sequence for CDR grafting). Following extensive analysis of all available germline sequences for both the variable heavy and light chain, germline candidates are selected based on their homology to the mouse/donor mAbs, and the CDRs from the mouse/donor progenitor mAbs were used to replace native canine CDRs. The objective is always to retain high affinity and eventual in vivo efficacy if being used as a therapeutic. Using canine antibody frameworks will generally minimize the potential of immunogenicity in vivo when administered to a dog. In some instances, however, framework region (FR) residues of the canine immunoglobulin are replaced by corresponding non-canine residues when reduced affinity or function is observed. Back mutation of selected target framework residues to the corresponding donor residues might be required to restore and or improved affinity, as noted. Structure-based methods may also be employed for caninization and affinity maturation. as described in U.S. Pat. No. 7,261,890. The above description uses canine as the target species and mouse as the donor species. Speciated antibodies are not limited to these targets and donors. Felines, and the like can be used as target species.

Another challenge for developing therapeutic antibodies targeting proteins is that epitopes on the homologous protein in a different species are frequently different, and the potential for cross-reactivity with other proteins is also different. As a consequence, antibodies have to be made, tested and developed for the specific target in the particular species to be treated. Antibody binding between homologous targets in different species is unpredictable and requires testing and evaluation of efficacy.

Antibodies target an antigen through its binding of a specific epitope on an antigen by the interaction with the variable region of the antibody molecule. Furthermore, antibodies have the ability to mediate, inhibit (as in the case of the antagonistic anti-TGFβ antibody of the present invention) and/or initiate a variety of biological activities. There are a wide range of functions for therapeutic antibodies, for example, antibodies can modulate receptor-ligand interactions as agonists or antagonists. Antibody binding can initiate intracellular signaling to stimulate cell growth, cytokine production, or apoptosis. Antibodies can deliver agents bound to the Fc region to specific sites. Antibodies also elicit antibody-mediated cytotoxicity (ADCC), complement-mediated cytotoxicity (CDC), and phagocytosis through the binding of the Fc region of the antibody to respective molecules in the cell which elicit ADCC, CDC etc. There are also antibodies that have been altered where the ADCC, CDC, C1q binding and phagocytosis functions have been eliminated. In one embodiment, the present invention provides an antibody comprising alterations in the Fc region of the antibody that alters effector function of said antibody. The present invention further provides cells and cell lines expressing antibodies of the invention. Representative host cells include bacterial, yeast, mammalian and human cells, such as CHO cells, HEK-293 cells, HeLa cells, CV-1 cells, and COS cells. Methods for generating a stable cell line following transformation of a heterologous construct into a host cell are well known in the art. Representative non-mammalian host cells include insect cells (Potter et al. (1993) Int. Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, monoclonal, chimeric, species specific and speciated antibodies, which have been modified by, ex., deleting, adding, or substituting other portions of the antibody, ex. the constant region, are also within the scope of the invention. For example, an antibody can be modified as follows: (i) by deleting the constant region; (ii) by replacing the constant region with another constant region, ex., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others. In one embodiment of the present invention the antibody of the invention comprises an altered Fc region that alters effector function of the antibody. In some embodiments of the present invention the Fc region of the antibody of the invention has been replaced, modified or removed.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see ex., EP388151 A1, U.S. Pat. Nos. 5,624,821 and 5,648,260, the contents of all of which are hereby incorporated by reference).

For example, it is possible to alter the affinity of an Fc region of an antibody for an FcR (ex. Fc.gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see ex., U.S. Pat. No. 5,624,821). The antibody or binding fragment thereof may be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (ex., methotrexate, 6-mercaptopurine, 6-thioguanine, cytarabine, and 5-fluorouracil decarbazine), alkylating agents (ex., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (ex., daunorubicin and doxorubicin), antibiotics (ex., dactinomycin, bleomycin, mithramycin, and anthramycin), and anti-mitotic agents (ex., vincristine and vinblastine). Techniques for conjugating such moieties to proteins are well known in the art.

### Compositions, Derived Compositions, and Methods of Making the Compositions

This invention encompasses compositions, including pharmaceutical compositions, comprising antibodies (“antigen binding proteins”, “antibody fragments”, “antagonist antibodies” and the like as used interchangeably herein), polypeptides and polynucleotides comprising sequences encoding antibodies or polypeptides of the invention.

As used herein, compositions comprise one or more antibodies or antigen binding polypeptides that bind to one or more of the TGFβ proteins, and/or one or more polynucleotides comprising sequences encoding one or more antibodies or polypeptides that bind to one or more of the TGFβ proteins. These compositions may further comprise suitable excipients, such as pharmaceutically/veterinary acceptable excipients including buffers, which are well known in the art. The invention also encompasses isolated antibody, polypeptide and polynucleotide embodiments. The invention also encompasses substantially pure antibody, polypeptide and polynucleotide embodiments.

In one or more embodiments, the present invention provides an isolated and recombinant antibody that binds to TGFβ1, TGFβ2 and TGFβ3 proteins, wherein the variable heavy chain comprises amino acid sequence having at least about 85%, 86%, 87%, 88%, 89%, 90%, 91%, 92%, 93%, 94%, 95%, 96%, 97%, 98% or 99% sequence identity to the amino acid sequence of the antibody of the invention as described herein and wherein the variable light chain comprises amino acid sequence having at least about 85%, 86%, 87%, 88%, 89%, 90%, 91%, 92%, 93%, 94%, 95%, 96%, 97%, 98% or 99% sequence identity to the amino acid sequence comprising the antibody of the invention as described herein and any variants thereof having one or more conservative amino acid substitutions in at least one of CDR1, CDR2 or CDR3 within any of the variable light or variable heavy chains of said antibody.

The present invention provides for recombinant antibodies, in some embodiments described herein, monoclonal antibodies, and antibody fragments and their uses in clinical administrations and scientific procedures, including diagnostic procedures. With the use of methods of molecular biology and recombinant technology, it is possible to produce an antibody and antibody-like molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with assembly of the synthesized chains to form active tetrameric (H2L2) structures with affinity for specific epitopes and antigenic determinants. This has permitted the ready production of antibodies having sequences characteristic of neutralizing antibodies from different species and sources.

Regardless of the source of the antibodies, how they are constructed, or how they are synthesized, in vitro or in vivo, using transgenic animals, large cell cultures of laboratory or commercial size, using transgenic plants, or by direct chemical synthesis employing no living organisms at any stage of the process, all antibodies have a similar overall 3-dimensional structure. This structure is often given as H2L2 and refers to the fact that antibodies commonly comprise two light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets.

As used herein, the term “antigen binding region” refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. Within the variable regions of the H or L chains that provide for the antigen binding regions are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have three CDR regions, each non-contiguous with the others. In all mammalian species, antibody peptides contain constant (i.e., highly conserved) and variable regions, and, within the latter, there are the CDRs and the so-called “framework regions” made up of amino acid sequences within the variable region of the heavy or light chain but outside the CDRs.

The present invention further provides a vector including at least one of the nucleic acids described above. Because of the degeneracy of the genetic code, more than one codon can be used to encode a particular amino acid. Using the genetic code, one or more different nucleotide sequences can be identified, each of which would be capable of encoding the amino acid. The probability that a particular oligonucleotide will, in fact, constitute the actual encoding sequence can be estimated by considering abnormal base pairing relationships and the frequency with which a particular codon is actually used (to encode a particular amino acid) in eukaryotic or prokaryotic cells expressing an anti-TGFβ antibody or portion. Such “codon usage rules” are disclosed by Lathe, et al., 183 J. Molec. Biol. 1-12 (1985). Using the “codon usage rules” of Lathe, a single nucleotide sequence, or a set of nucleotide sequences that contains a theoretical “most probable” nucleotide sequence capable of encoding anti-TGFB sequences can be identified. It is also intended that the antibody coding regions for use in the present invention could also be provided by altering existing antibody genes using standard molecular biological techniques that result in variants (agonists) of the antibodies and peptides described herein. Such variants include, but are not limited to deletions, additions and substitutions in the amino acid sequence of the anti-TGFB antibodies or peptides.

### Antibody Derivatives

Included within the scope of this invention are antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

Derivatives also include radioactively labeled monoclonal antibodies that are labeled. For example, with radioactive iodine (251,1311), carbon (4C), sulfur (35S), indium, tritium (H³) or the like; conjugates of monoclonal antibodies with biotin or avidin, with enzymes, such as horseradish peroxidase, alkaline phosphatase, beta-D-galactosidase, glucose oxidase, glucoamylase, carboxylic acid anhydrase, acetylcholine esterase, lysozyme, malate dehydrogenase or glucose 6-phosphate dehydrogenase; and also conjugates of monoclonal antibodies with bioluminescent agents (such as luciferase), chemoluminescent agents (such as acridine esters) or fluorescent agents (such as phycobiliproteins).

Another derivative bifunctional antibody of the present invention is a bispecific antibody, generated by combining parts of two separate antibodies that recognize two different antigenic groups. This may be achieved by crosslinking or recombinant techniques. Additionally, moieties may be added to the antibody or a portion thereof to increase half-life in vivo (ex., by lengthening the time to clearance from the blood stream. Such techniques include, for example, adding PEG moieties (also termed pegilation), and are well-known in the art. See U.S. Patent. Appl. Pub. No. 20030031671.

### Recombinant Expression of Antibodies

In some embodiments, the nucleic acids encoding the antibodies of the invention are introduced directly into a host cell, and the cell is incubated under conditions sufficient to induce expression of the encoded antibody. After the subject nucleic acids have been introduced into a cell, the cell is typically incubated, normally at 37° C., sometimes under selection, for a period of about 1-24 hours in order to allow for the expression of the antibody. In one embodiment, the antibody is secreted into the supernatant of the media in which the cell is growing. Traditionally, monoclonal antibodies have been produced as native molecules in murine hybridoma lines. In addition to that technology, the present invention provides for recombinant DNA expression of monoclonal antibodies. This allows the production of said antibodies, as well as a spectrum of antibody derivatives and fusion proteins in a host species of choice.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as anti-TGFβ antibodies or antibody fragments in recoverable amounts. The precise nature of the regulatory regions needed for gene expression may vary from organism to organism, as is well known in the analogous art.

The present invention accordingly encompasses the expression of an anti-TGFβ antibody or, in either prokaryotic or eukaryotic cells. Suitable hosts include bacterial or eukaryotic hosts including bacteria, yeast, insects, fungi, bird and mammalian cells either in vivo, or in situ, or host cells of mammalian, insect, bird or yeast origin. The mammalian cell or tissue may be of human, primate, hamster, rabbit, rodent, cow, pig, sheep, horse, goat, dog or cat origin, but any other mammalian cell may be used without limitation.

The expression vector carrying a chimeric, speciated antibody construct or anti-TGFβ antibody of the present invention can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et at, 240 Science 1538 (1988) or other techniques known to one of skill in the art without limitation For long-term, high-yield production of recombinant antibodies, stable expression may be used.

For example, cell lines, which stably express the antibody molecule may be engineered. Rather than using expression vectors which contain origins of replication, host cells can be transformed with immunoglobulin expression cassettes and a selectable marker. Following the introduction of the foreign DNA, engineered cells may be allowed to grow in enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds/components that interact directly or indirectly with the antibody molecule.

Once the antibody of the invention has been produced, it may be purified by any method known in the art for purification of an immunoglobulin molecule, for example but without limitation, by chromatography (ex. ion exchange, affinity, particularly affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

### Pharmaceutical and Veterinary Applications

The anti-TGFβ antibody or antibody fragments of the invention as described herein can be used for example in the treatment of TGFβ related disorders in canines and felines. More specifically, the invention further provides for a pharmaceutical composition comprising a pharmaceutically acceptable carrier or diluent and, as active ingredient, an antibody or antibody fragment per the invention. The antibody can be a chimeric, heterochimeric, caninized or felinized antibody to accommodate a different non-human species. Intact immunoglobulins or their binding fragments, are also envisioned. The antibody and pharmaceutical compositions thereof of this invention are useful for parenteral administration, ex., subcutaneously, intramuscularly or intravenously.

In some desired embodiments, the antibodies of the invention are administered by parenteral injection. For parenteral administration, anti-TGFβ antibodies or fragments can be formulated as a solution, suspension, emulsion or lyophilized powder in association with a pharmaceutically acceptable parenteral vehicle. For example, the vehicle may be a solution of the antibody or a cocktail thereof dissolved in an acceptable carrier, such as, but not limited to, an aqueous carrier such vehicles are water, saline, Ringer's solution, dextrose solution, trehalose or sucrose solution, or serum albumin, glycine and the like. Liposomes and non-aqueous vehicles such as fixed oils can also be used. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjustment agents and the like, for example sodium acetate, sodium chloride, potassium chloride, calcium chloride, sodium lactate, etc. The concentration of antibody in these formulations can vary widely, for example from less than about 0.5%, usually at or at least about 1% to as much as 15% or 20% by weight and will be selected primarily based on fluid volumes, viscosities, etc., in accordance with the particular mode of administration selected. The vehicle or lyophilized powder can contain additives that maintain isotonicity (ex., sodium chloride, mannitol) and chemical stability (ex., buffers and preservatives). The formulation is sterilized by commonly used techniques. Actual methods for preparing parenterally administrable compositions will be known or apparent to those skilled in the art and are described in more detail in, for example, REMINGTON'S PHARMA. SCI. (15th ed., Mack Pub. Co., Easton, Pa., 1980).

The antibodies of this invention can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of antibody activity loss and that use levels may have to be adjusted to compensate. The antibody compositions of the present invention may provide a cocktail thereof can be administered for prevention of recurrence and/or therapeutic treatments for existing disease. Suitable pharmaceutical carriers are described in the most recent edition of REMINGTON'S PHARMACEUTICAL SCIENCES, a standard reference text in this field of art among other references well known to those of skill in the art. In therapeutic application, compositions are administered to a subject already suffering from a disease or condition, in an amount sufficient to cure or at least partially arrest or alleviate the disease or conditions and its complications. An amount adequate to accomplish this is defined as a “therapeutically effective dose” or a “therapeutically effective amount”.

The dosage administered will, of course, vary depending upon known factors such as the pharmacodynamic characteristics of the particular agent, and its mode and route of administration; age, health, and weight of the recipient; nature and extent of symptoms kind of concurrent treatment, frequency of treatment, and the effect desired.

As a non-limiting example, treatment of TGFβ-related pathologies in dogs and cats can be provided in the dosage range as needed. Example antibodies for canine or feline therapeutic use are high affinity antibodies, and fragments, regions and derivatives thereof having potent in vivo anti-TGFB activity, according to the present invention. Single or multiple administrations of the compositions can be carried out with dose levels and pattern being selected by the treating veterinarian. In any event, the pharmaceutical formulations should provide a quantity of the antibody(ies) of this invention sufficient to effectively treat the subject.

### Diagnostic Applications

The present invention also provides the above anti-TGFβ antibodies for use in diagnostic methods for detecting TGFβ in species, particularly canines and felines known to be or suspected of having an TGFβ related disorder. Anti-TGFβ antibodies of the present invention are useful for immunoassays which detect or quantitate one or more TGFβ, or anti-TGFβ antibodies, in a sample. An immunoassay for TGFβ typically comprises incubating a clinical or biological sample in the presence of a detectably labeled high affinity (or high avidity) anti-TGFβ antibody of the present invention capable of selectively binding to TGFβ and detecting the labeled peptide or antibody which is bound in a sample. Various clinical assay procedures are well known in the art. Such samples include tissue biopsy, blood, serum, and fecal samples, or liquids collected from animal subjects and subjected to ELISA analysis as known to those of skill in the art.

“Solid phase support” or “carrier” refers to any support capable of binding peptide, antigen, or antibody. Well-known supports, or carriers, include glass, polystyrene, polypropylene, polyethylene, polyvinylidenefluoride (PVDF), dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to one or more TGFβ proteins or an anti-TGFβ antibody. Thus, the support configuration can be spherical, as in a bead, or cylindrical, as in the inside surface of a test tube, or the external surface of a rod. Alternatively, the surface can be flat, such as a sheet, culture dish, test strip, etc. For example, supports may include polystyrene beads. Those skilled in the art will know many other suitable carriers for binding antibody, peptide or antigen, or can ascertain the same by routine experimentation. Well known method steps can determine binding activity of a given lot of an anti-TGFB peptide and/or antibody. Those skilled in the art can determine operative and optimal assay conditions by routine experimentation.

Detectably labeling an TGFβ-specific peptide and/or antibody can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example but not limited to, spectrophotometric, fluorometric or by visual means. Enzymes which can be used to detectably label the TGFβ-specific antibodies of the present invention include, but are not limited to, malate dehydrogenase, staphylococcal nuclease, delta5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. By radioactively labeling the TGFβ-specific antibodies, it is possible to detect TGFβ through the use of a radioimmunoassay (RIA). The radioactive isotope can be detected by such means as the use of a gamma counter or a scintillation counter or by autoradiography. Isotopes which are particularly useful for the purpose of the present invention include: ³H, ¹²⁵I, ¹³¹I, ³⁵S and ¹⁴C.

It is also possible to label the TGFβ-specific antibodies with a fluorescent compound. When the fluorescent labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine among those known to those of skill in the art. The TGFβ-specific antibodies can also be delectably labeled using fluorescence-emitting metals such a ¹²⁵Eu, or others of the lanthanide series. These metals can be attached to the TGFβ specific antibody using such metal chelating groups as diethylenetriaminepentaacetic acid (DTPA) or ethylenediamine-tetraacetic acid (EDTA).

The TGFβ-specific antibodies also can be detectably labeled by coupling to a chemiluminescent compound. The presence of the chemiluminescently labeled antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound can be used to label the TGFβ-specific antibody, portion, fragment, polypeptide, or derivative of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the TGFβ-specific antibody, portion, fragment, polypeptide, or derivative can be accomplished by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

For the purposes of the present invention, the TGFβ which is detected by the above assays can be present in a biological sample. Any sample containing TGFβ may be used. For example, the sample is a biological fluid such as, for example, blood, serum, lymph, urine, feces, inflammatory exudate, cerebrospinal fluid, amniotic fluid, a tissue extract or homogenate, and the like as well as any biopsy related material. The invention is not limited to assays using only these samples, however, it being possible for one of ordinary skill in the art, in light of the present specification, to determine suitable conditions which allow the use of other samples.

In situ detection can be accomplished by removing a histological specimen from an animal subject and providing the combination of labeled antibodies of the present invention to such a specimen. The antibody (or portion thereof) may be provided by applying or by overlaying the labeled antibody (or portion) to a biological sample. Through the use of such a procedure, it is possible to determine not only the presence of TGFβ but also the distribution of TGFβ in the examined tissue. Using the present invention, those of ordinary skill will readily perceive that any of a wide variety of histological methods (such as staining procedures) can be modified in order to achieve such in situ detection.

The antibody, fragment or derivative of the present invention can be adapted for utilization in an immunometric assay, also known as a “two-site” or “sandwich” assay. In a typical immunometric assay, a quantity of unlabeled antibody (or fragment of antibody) is bound to a solid support that is insoluble in the fluid being tested and a quantity of detectably labeled soluble antibody is added to permit detection and/or quantification of the ternary complex formed between solid phase antibody, antigen, and labeled antibody.

The antibodies may be used to quantitatively or qualitatively detect one or more TGFβ proteins in a sample or to detect presence of cells that express one or more of the TGFβ proteins. This can be accomplished by immunofluorescence techniques employing a fluorescently labeled antibody (see below) coupled with fluorescence microscopy, flow cytometric, or f1uorometric detection. For diagnostic purposes, the antibodies may either be labeled or unlabeled. Unlabeled antibodies can be used in combination with other labeled antibodies (second antibodies) that are reactive with the antibody, such as antibodies specific for canine immunoglobulin constant regions. Alternatively, the antibodies can be directly labeled. A wide variety of labels may be employed, such as radionuclides, fluors, enzymes, enzyme substrates, enzyme cofactors, enzyme inhibitors, ligands (particularly haptens), etc. Numerous types of immunoassays, such as those discussed previously are available and are well known to those skilled in the art. Importantly, the antibodies of the present invention may be helpful in diagnosing a TGFβ related disorder in canines, and felines. More specifically, the antibody/antibody of the present invention may identify the overexpression of TGFβ in companion animals. Thus, the antibody of the present invention may provide an important immunohistochemistry tool. The antibodies of the present invention may be used on antibody arrays, highly suitable for measuring gene expression profiles and other diagnostic tools well known to those of skill in the art.

### Kits

Also included within the scope of the present invention are kits for practicing the subject methods. The kits at least include one or more of the antibodies of the present invention, a nucleic acid encoding the same, or a cell containing the same. An antibody of the present invention may be provided, usually in a lyophilized form, in a container. The antibodies, which may be conjugated to a label or toxin, or unconjugated, are typically included in the kits with buffers, such as Tris, phosphate, carbonate, etc., stabilizers, biocides, inert proteins, ex., serum albumin, or the like. Generally, these materials will be present in less than 5% wt. based on the amount of active antibody, and usually present in total amount of at least about 0.001% wt. based again on the antibody concentration. Frequently, it will be desirable to include an inert extender or excipient to dilute the active ingredients, where the excipient may be present in from about, 1% to 99% wt. of the total composition. Where a second antibody capable of binding to the primary antibody is employed in an assay, this will usually be present in a separate vial. The second antibody is typically conjugated to a label and formulated in an analogous manner with the antibody formulations described above. The kit will generally also include a set of instructions for use

Before the present methods are described, it is to be understood that this invention is not limited to particular methods, and experimental conditions described, as such methods and conditions may vary yet are well known to those of skill in the art. It is also to be understood that the terminology used herein is for the purpose of describing particular embodiments only and is not intended to be limiting. The invention will now be described further by the non-limiting examples below.

### EXAMPLES

The present invention is further illustrated and supported by the following examples. However, these examples should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### Example 1

### Anti-TGFβ 1,2,3 Antibody

The anti-TGFβ1, 2, 3 GC1008 antibody (VH: SEQ ID NO. 74 and VH: SEQ ID NO. 75) was speciated using both caninization and felinization techniques as describes herein.

The six CDRs for the GC1008 monoclonal antibody are as follows:

TABLE 1

| SEQ | | |
|---|---|---|
| ID | | Amino Acid |
| NO: | Description | Sequence |
| 23 | heavy chain CDR #1 (CDR-H1) | GYTPSSNVIS |
| 33 | heavy chain CDR #2 (CDR-H2) | GVIPIVDIAN |
| | | YAQRPKGR |
| 39 | heavy chain CDR #3 (CDR-H3) | ASTLGLVLDA |
| | | MDY |
| 3 | light chain CDR #1 (CDR-K1) | RASQSL |
| 4 | light chain CDR #2 (CDR-K2) | GASSRA |
| 5 | light chain CDR #3 (CDR-K3) | QQYADSPIT |

### Construction of Recombinant Human: Feline Chimera

Antibody variable domains are responsible for antigen binding, and therefore grafting of the full variable domain of the GC1008 antibody onto a different constant region, for example a constant region from a different species, should have little or no impact on the antibody's ability to bind the feline TGFβ proteins. As such, expression vectors were designed to produce recombinant chimeric antibodies in mammalian expression systems. Chimeric antibodies described herein consist of the variable sequence (both CDR and framework) from the host species antibody grafted onto the respective heavy and light constant regions of an IgG molecule from a different species. For example, the variable region from the humanized antibody, ex., SEQ ID NOS: 79 and, SEQ ID NO. 80 and the heavy chain constant region from a feline species (amino acid SEQ ID NO. 75), which would be referred to herein as a human: feline chimera. To produce the desired chimeric antibodies, synthetic DNA sequences were constructed for the variable heavy (VH) and variable light (VL) sequences of selected antibodies which contain unique restriction endonuclease sites, Kozak consensus sequence and, an N-terminal secretion leader to facilitate expression and secretion of the recombinant antibody from a mammalian cell line.

For the human: feline GC1008 chimera, referred to herein as felchimGC1008, the human variable regions (SEQ ID NOS: 79 and 80) were cloned into a mammalian expression plasmid containing either the feline IgG heavy (SEQ ID NO: 75) or light chain constant regions (SEQ ID NO. 83). The plasmids encoding each heavy and light chain, under the control of the CMV promoter, were co-transfected into HEK 293 cells using standard methods (SEQ ID NO 1 and SEQ ID NO. 2). Following six days of expression, chimeric mAbs were purified from 50 ml of transiently transfected HEK293FS cell supernatants using MabSelect Sure protein A resin (GE Healthcare, Uppsala, Sweden) according to standard methods for protein purification. Eluted fractions were neutralized, concentrated to ˜0.5-1.0 mL using a 10,000 nominal MW cutoff Amicon Ultra centrifugal device (Millipore Sigma, Burlington, Mass.), dialyzed overnight at 4° C. in 20 mM sodium Acetate pH 5.0, 85 g/L sucrose, +/−0.05 g/L EDTA, and stored at 4° C. for further use.

### Caninization of Antibody GC1008

The generation of anti-drug antibodies (ADAs) has been associated with loss of efficacy for any biotherapeutic protein, including monoclonal antibodies. Speciation of monoclonal antibodies can reduce the propensity for mAbs to be immunogenic, although examples of immunogenic fully human mAbs and non-immunogenic chimeric mAbs can be found. To help mitigate risks associated with ADA formation for the GC1008 monoclonal antibody provided herein, a caninization strategy was employed. This caninization strategy is based on identifying the most appropriate canine germline antibody sequence for CDR grafting. Following extensive analysis of available canine germline sequences for both the variable heavy and light chains, germline candidates were selected based on their homology to the framework regions of the GC1008 antibody variable regions, and the CDRs (VH SEQ ID NOs 23,33, and 39, and VL SEQ ID NOs 3,4,5) were used to replace native canine CDRs. The objective was to retain high affinity and cell-based activity using canine antibody frameworks to minimize the potential of immunogenicity in vivo.

Synthetic nucleotide constructs representing the caninized variable heavy and light chains for the caninized GC1008 antibody were made. Caninization efforts with the GC1008 antibody initially focused on synthetic nucleotide constructs representing four canine variable heavy chains (VH2, 3, 4 and 6) and four canine kappa light chains (VL1-4) were chosen for the initial caninization. Following subcloning of each variable chain into plasmids containing the respective canine heavy (SEQ ID NO: 77) or kappa (SEQ ID NO: 81) constant region plasmids were co-transfected for antibody expression in HEK 293 cells in all possible combinations. to make numerous caninized antibody constructs. All but two of the caninized constructs expressed in transient HEK293 expression systems. After expression, binding to all three TGFβ isoforms was assessed. See below. Surprisingly, no in vitro binding or functional activity was observed. Amino acids in the CDR and framework regions were mutated, inserted or deleted to fine tune the mAb binding interface to the target isotypes. Pre-determined standard human complementarity-determining regions grafted on canine frameworks significantly reduced affinity to the target isotypes hence, additional engineering was required to regain comparable affinities to the targets of the fully species template mAb. The VH and VL re-engineered are denoted by * in Table 2 below.

TABLE 2

| | |
|---|---|
| Heavy or | SEQ ID NO for |

| Alias | light chain | Nucleic acid | Amino Acid |
|---|---|---|---|
| VH2 | Heavy | 55 | 42 |
| VH6 | Heavy | 56 | 43 |
| VH3 | Heavy | 57 | 44 |
| VH4 | Heavy | 58 | 45 |
| VH4.11* | Heavy | 59 | 46 |
| VH5.10* | Heavy | 60 | 47 |
| VH4.5* | Heavy | 61 | 48 |
| VH3.1* | Heavy | 62 | 49 |
| VH3.5* | Heavy | 63 | 50 |
| VH3.6* | Heavy | 64 | 51 |
| VH4.7* | Heavy | 65 | 52 |
| VH5.6* | Heavy | 66 | 54 |
| VL1 | Light | 71 | 67 |
| VL2 | Light | 72 | 68 |
| VL3 | Light | 73 | 69 |
| VL4 | Light | 74 | 70 |

### Felinization of GC1008

As with the caninization of the GC1008 antibody, felinized antibodies were generated by taking the same CDR region sequences used for caninization and incorporating them with feline variable framework sequences. Feline databases were searched for similar frameworks to the chimeric and/or caninized antibodies to identify feline germlines to investigate. Initially, two heavy chain frameworks and four light chain frameworks were selecting, resulting in the production of the following felinized variable regions:

Following subcloning of each variable chain into plasmids containing the respective feline heavy (SEQ ID NO: 75) or kappa (SEQ ID NO: 83) constant region plasmids were co-transfected for antibody expression in HEK 293 cells. Co-transfections were performed to give combinations of heavy and light chains. Binding and functional assays (described below) were performed on the original VH and VL in the caninized and felinized versions. Surprisingly, no in vitro binding or functional activity was observed. Amino acids in the CDR and framework regions were mutated, inserted or deleted to fine tune the mAb binding interface to the target isotypes. Pre-determined standard human complementarity-determining regions grafted on feline frameworks significantly reduced affinity to the target isotypes hence, additional engineering was required to regain comparable affinities to the targets of the fully species template mAb. The VH and VL re-engineered are denoted by * in table 3 below.

TABLE 3

| | |
|---|---|
| Heavy or | SEQ ID NO for |

| Alias | light chain | Nucleic acid | Amino Acid |
|---|---|---|---|
| VH8 | Heavy | 97 | 87 |
| VH9 | Heavy | 98 | 88 |
| VH4 | Heavy | 96 | 86 |
| VH3 | Heavy | 95 | 85 |
| VH3.2* | Heavy | 99 | 89 |
| VH2.1* | Heavy | 100 | 90 |
| VH6.1* | Heavy | 101 | 91 |
| VH2.2* | Heavy | 102 | 92 |
| VH2.8* | Heavy | 103 | 93 |
| VH3.8* | Heavy | 104 | 94 |
| VL1 | Light | 14 | 6 |
| VL2 | Light | 15 | 7 |
| VL3 | Light | 16 | 8 |
| VL4 | Light | 17 | 9 |
| VL5 | Light | 18 | 10 |
| VL6 | Light | 19 | 11 |
| VL7 | Light | 20 | 12 |
| VL8 | Light | 21 | 13 |

### In Vitro Binding and Functional Assays

Affinity and cell-based potency of felchimGC1 008 and all caninized and felinized antibodies04H09 and chi64H09 were assessed using surface plasmon resonance (SPR). To characterize the affinity with which candidate monoclonal antibodies (mAbs) bind TGFβ1, 2 and 3, surface plasmon resonance (SPR) was evaluated using a Biacore T200 system (Biocore Life Sciences (GE Healthcare), Uppsala, Sweden). To avoid affinity differences associated with differential surface preparation that can occur when immobilizing antibodies to surfaces, TGFβ1, TGFβ2, and TGFβ3 (R&D Systems) were directly conjugated to the individual surface. Immobilization was obtained by amine coupling 5 pg/mL using N-hydroxysuccinimide (NHS)/1-Ethyl-3-(3-dimethylaminopropyl) carbodiimide (EDO) chemistry. Chips were quenched with ethanolamine and the affinity with which all candidate mAbs bound to the immobilized TGFβ was evaluated. All curves were fit to a 1:1 model. Affinities <10-11 are below the lower limit of quantitation of detection for the instrument.

TABLE 4
Caninized mAb VH/VL combinations in vitro binding

| | VH | VL | TGFβ1 | TGFβ2 | TGFβ3 |
|---|---|---|---|---|---|
| | SEQ | SEQ | KD | KD | KD |
| Sample Name | ID NO. | ID NO. | (M) | (M) | (M) |
| VH2 - VL4 | 42 | 70 | None | None | None |
| VH6 - VL3 | 43 | 69 | None | None | None |
| VH3 - VL2 | 44 | 68 | None | None | None |
| VH4 - VL2 | 45 | 68 | None | None | None |
| VH4.11 - VL3 | 46 | 69 | 7.14E−09 | 5.15E−09 | 2.50E−09 |
| VH5.10 - VL3 | 47 | 69 | 5.23E−09 | 3.57E−09 | 1.50E−09 |
| VH4.5 - VL1 | 48 | 67 | 2.57E−09 | 1.56E−09 | 4.75E−10 |
| VH3.1 - VL2 | 49 | 68 | 5.60E−09 | 3.37E−09 | 1.12E−09 |
| (ZTS-122) | | | | | |
| VH3.5 - VL2 | 50 | 68 | 4.37E−09 | 3.56E−09 | 1.91E−09 |
| VH4.5 - VL2 | 48 | 68 | 2.30E−09 | 1.40E−09 | 4.30E−10 |
| (ZTS-207) | | | | | |
| VH3.6 - VL2 | 51 | 68 | 4.39E−09 | 3.42E−09 | 1.49E−09 |
| VH4.7 - VL2 | 52 | 68 | 2.61E−09 | 1.99E−09 | 1.20E−09 |
| VH4.11 - VL2 | 46 | 68 | 4.98E−09 | 3.77E−09 | 2.10E−09 |
| VH5.6 - VL2 | 54 | 68 | 3.68E−09 | 2.60E−09 | 1.67E−09 |
| VH3.1 - VL3 | 49 | 69 | 7.52E−09 | 3.91E−09 | 9.81E−10 |

TABLE 5
Felinized mAb VH/VL combinations in vitro binding

| | TGFb1 K | TGFb2 KD | TGFb3 KD |
|---|---|---|---|
| Sample Name | (M) | (M) | (M) |
| felchimGC1008 | 2.58E−09 | 1.88E−09 | 2.35E−09 |
| VH8 - VL2 | None | None | None |
| VH9 - VL3 | None | None | None |
| VH4 - VL4 | None | None | None |
| VH3 - VL5 | None | None | None |
| VH2.1 - VL3 | 2.89E−09 | 1.91E−09 | 2.6E−09 |
| VH6.1 - VL3 | 3.54E−09 | 2.99E−09 | 3.57E−09 |
| VH2.2 - VL3 | 3.31E−09 | 2.39E−09 | 3.49E−09 |
| VH3.2 - VL3 | 6.6E−10 | 7.51E−10 | 1.44E−10 |
| VH2.8 - VL6 | 2.95E−09 | 1.72E−09 | 2.41E−09 |
| VH2.2 - VL7 | 4.95E−09 | 2.16E−09 | 3.52E−09 |
| VH3.2 - VL7 | 8.57E−10 | 5.89E−10 | 4.34E−10 |
| VH3.8 - VL7 | 3.07E−09 | 1.94E−09 | 6.35E−10 |
| VH2.1 - VL8 | 3.31E−09 | 3.41E−09 | 1.06E−09 |
| VH3.2 - VL8 | 9.71E−10 | 2.13E−09 | 4.66E−11 |
| VH3.2 - VL1 | 3.33E−10 | 1.84E−09 | 5.1E−12 |

To assay for potency a primary cell-based assay measuring inhibition of TGFβ1-induced SMAD3 phosphorylation in canine mitral valve interstitial cells (CMVICs). For this assay, TGFβ1, 2, or 3 was added to the cells with or without antibody and SMAD3 signaling was determined via AlphaLISA detection kit. The affinity of all mAbs to TGFβ1, 2, and 3 surfaces and potency of each antibody are shown in Tables 6 and 7 (below) (K_{D} data and CMVIC data, respectively).

TABLE 6
Caninized mAb VH/VL combinations functional inhibition assays

| | TGFb1_IC50 | TGFb2_IC50 | TGFb3_IC50 |
|---|---|---|---|
| Sample Name | ug/mL | ug/mL | ug/mL |
| VH2 - VL4 | | | |
| VH6 - VL3 | | | |
| VH3 - VL2 | | | |
| VH4 - VL2 | | | |
| VH4.11 - VL3 | 1.011 | 16.720 | 0.575 |
| VH5.10 - VL3 | 0.232 | 5.471 | 0.113 |
| VH4.5 - VL1 | 0.122 | 0.609 | 0.062 |
| VH3.1 - VL2 | 0.061 | 0.508 | 0.092 |
| VH3.5 - VL2 | 0.083 | 9.408 | 0.981 |
| VH4.5 - VL2 | 0.060 | 0.285 | 0.020 |
| VH3.6 - VL2 | 0.057 | 0.459 | 0.203 |
| VH4.7 - VL2 | 0.071 | 0.810 | — |
| VH4.11 - VL2 | 0.164 | 8.484 | 0.375 |
| VH5.6 - VL2 | 0.019 | 0.691 | 0.056 |
| VH3.1 - VL3 | 0.134 | 0.954 | 0.034 |

TABLE 7
Felinized mAb VH/VL combinations in
vitro functional inhibitions assays

| | TGFb1_IC50 | TGFb2_IC50 | TGFb3_IC50 |
|---|---|---|---|
| Sample Name | ug/mL | ug/mL | ug/mL |
| felchimGC1008VH | 0.1762 | 0.1668 | 0.05296 |
| VH8 - VL2 | — | — | — |
| VH9 - VL3 | — | — | — |
| VH4 - VL4 | — | — | — |
| VH3 - VL5 | — | — | — |
| VH2.1 - VL3 | 0.597 | 0.277 | 0.132 |
| VH6.1 - VL3 | 0.425 | 0.223 | 0.014 |
| VH2.2 - VL3 | 0.240 | 1.197 | 0.000 |
| VH3.2 - VL3 | 0.132 | 0.140 | 0.023 |
| VH2.8 - VL6 | 0.969 | 0.547 | 0.000 |
| VH2.2 - VL7 | 4.587 | 20.600 | 2.203 |
| VH3.2 - VL7 | 0.139 | 0.048 | 0.018 |
| VH3.8 - VL7 | 1.357 | 2.667 | 0.204 |
| VH2.1 - VL8 | 0.266 | 0.031 | 0.124 |
| VH3.2 - VL8 | 0.072 | 0.037 | 0.008 |
| VH3.2 - VL1 | 0.797 | 0.190 | 0.053 |

### Example 2

### Pharmacokinetics of ZTS-122 and ZTS-207

A study to determine the subcutaneous pharmacokinetics of caninized ZTS-122 (VH3.1 and VL2 [SEQ ID NO. 49 and SEQ ID NO. 68, respectively]) and ZTS-207 (VH4.5-VL2 [SEQ ID NO. 48 and SEQ ID NO. 68, respectively]) was undertaken. Each molecule was given to 4 dogs at a 1 mg/kg subcutaneous administration on days 0 and 28. Plasma samples were collected at pre-dose, days 1, 3, 7, 10, 14, 21, 28, 29, 31, 35, 38, 43, 49 and 56 from the animals. Exposure of each monoclonal antibody in plasma was assessed using ligand binding methods. Their pharmacokinetic properties were evaluated using noncompartmental analysis for period 1 (dose on day 0) and period 2 (dose on day 28). Please see Table 8 (below).

TABLE 8

| | | | | |
|---|---|---|---|---|
| mAb | Cmax | Tmax | T½ | AUC |
| (1 mg/kg) | μg/mL | Days | Days | μg*Days/mL |

Period 1

| | | | | |
|---|---|---|---|---|
| ZTS-122 | 4.07 ± 1.07 | 5.8 ± 3.4 | 7.81 ± 1.34 | 67.2 ± 17.8 |
| ZTS-207 | 6.24 ± 1.13 | 5 ± 2.3 | 21.4 ± 8.35 | 116 ± 30.8 |

Period 2

| | | | | |
|---|---|---|---|---|
| ZTS-122 | 7.81 ± 1.38 | 2.3 ± 1.2 | 9.63 ± 1.45 | 107 ± 14.8 |
| ZTS-207 | 9.17 ± 1.86 | 3.5 ± 2.5 | 12.1 ± 2.11 | 154 ± 36.7 |

We claim:

1. A caninized antibody that specifically binds to canine TGFβ1, TGFβ2 and TGFβ3, comprising heavy chain complimentarity determining regions (CDRs) comprising SEQ ID NO. 23; SEQ ID NO. 33 and SEQ ID NO. 39 and light chain complimentarity determining regions (CDRs) comprising SEQ ID NO. 3, SEQ ID NO. 4 and SEQ ID NO. 5 and variants thereof.

2. The caninized antibody of claim 1, further comprising a canine IgGB constant region comprising an amino acid sequence having at least about 95% sequence identity to SEQ ID NO. 77.

3. A caninized antibody that specifically binds to canine TGFβ1, TGFβ2 and TGFβ3 comprising a heavy chain variable region (VH) having at least about 95% sequence identity to the amino acid sequences selected from the group consisting of: SEQ ID NOs. 42-54; and a light chain variable region (VL) having at least about 95% sequence identity to the amino acid sequences selected from the group consisting of: SEQ ID NOs 67-70.

4. The caninized antibody of claim 3 comprising a heavy chain variable region (VH) having at least about 95% sequence identity to the amino acid sequence comprising SEQ ID NO. 49; and comprising a light chain variable region (VL) having at least about 95% sequence identity to the amino acid sequence comprising SEQ ID NO. 68.

5. The caninized antibody of claim 3 comprising a heavy chain variable region (VH) comprising SEQ ID NO. 49; and comprising a light chain variable region (VL) comprising SEQ ID NO. 68.

6. The caninized antibody of claim 3 comprising a heavy chain variable region (VH) having at least about 95% sequence identity to the amino acid sequence comprising SEQ ID NO. 48; and comprising a light chain variable region (VL) having at least about 95% sequence identity to the amino acid sequence comprising SEQ ID NO. 68.

7. The caninized antibody of claim 3 comprising a heavy chain variable region (VH) comprising SEQ ID NO. 49; and comprising a light chain variable region (VL) comprising SEQ ID NO. 68.

8. A pharmaceutical composition comprising a therapeutically effective amount of the antibody of either of claim 1 or 3 and a pharmaceutically acceptable carrier.

9. A method of treating a canine for a TGFβ related disorder by administering to said canine a therapeutic amount of the pharmaceutical composition of claim 8.

10. The method of claim 9 wherein the TGFβ related disorder is selected from the group consisting of: fibrosis disorder, connective tissue disorder, bone disorders and cell proliferation disorders.

11. The method of claim 10 wherein the TGFβ related disorder comprises a fibrosis disorder.

12. The method of claim 11 wherein the fibrosis disorder is selected from the group consisting of kidney fibrosis/chronic kidney disease; pulmonary fibrosis; cirrhosis of the liver; glial scarring; and systemic sclerosis/scleroderma.

13. The method of claim 12 wherein the TGFβ disorder is kidney fibrosis/chronic kidney disease.

14. A method of inhibiting TGFβ1, 2 and 3 activity in a canine by administering the pharmaceutical composition of claim 8.

15. An isolated nucleic acid sequence having at least about 95% sequence identity to the nucleic acid sequence encoding the antibody of either claim 1 or 3 and any variants thereof having one or more nucleic acid substitutions resulting in conservative amino acid substitutions.

16. An isolated nucleic acid sequence encoding the antibody of either claim 1 or 3 wherein said sequence comprises a nucleotide sequence encoding the VH having 95% sequence identity to SEQ ID NO. 55-66 and a nucleotide sequence encoding the VL having 95% sequence identity to SEQ ID NO. 71-74 and any variants thereof having one or more nucleic acid substitutions resulting in conservative amino acid substitutions.

17. An isolated nucleic acid sequence encoding the antibody of claim 3 wherein said sequence comprises a nucleotide sequence encoding the VH having 95% sequence identity to SEQ ID NO. 62 and a nucleotide sequence encoding the VL having 95% sequence identity to SEQ ID NO. 72 and any variants thereof having one or more nucleic acid substitutions resulting in conservative amino acid substitutions.

18. An isolated nucleic acid sequence encoding the antibody of claim 3 wherein said sequence comprises a nucleotide sequence encoding the VH having 95% sequence identity to SEQ ID NO. 61 and a nucleotide sequence encoding the VL having 95% sequence identity to SEQ ID NO. 72 and any variants thereof having one or more nucleic acid substitutions resulting in conservative amino acid substitutions.

19. A vector comprising the nucleic acid sequence of either claim 17 or claim 18.

20. A host cell comprising the nucleic acid sequence of claim 19.

21. A host cell comprising the vector of claim 20.

22. A host cell that produces the antibody of either claim 1 or 3.

23. A method of producing the antibody of either claim 1 or 3 comprising culturing the host cell of any one of claims 20-22 under conditions that result in production of the antibody and isolating the antibody from the host cell or culture medium of the host cell.
